(19) United States
(12) Patent Application Publication
(10) Pub. No.: US 2008/0299555 A1
(43) Pub. Date: Dec. 4, 2008

(54) Multicolor chromogenic detection of biomarkers

(76) Inventors: Hiroaki Nitta, Oro Valley, AZ (US); Thomas M. Grogan, Tucson, AZ (US)

Correspondence Address: VENTANA MEDICAL SYSTEMS, INC.;ATTENTION: LEGAL DEPARTMENT, 1910 INNOVATION PARK DRIVE, TUCSON, AZ, 85755, US

(21) Appl. No.: 11/809,024

(22) Filed: May 30, 2007

(51) Int. Cl. C12Q 1/68; C12Q 1/00; C12Q 1/26; C12Q 1/28; G01N 33/53

(52) U.S. Cl. 435/6; 435/4; 435/25; 435/28; 435/7.9

(57) ABSTRACT

The present invention provides compositions, kits, assembles of articles and methodology for detecting multiple target molecules in a sample, such as in a tissue sample. In particular, site-specific deposition of elemental metal is used in conjunction with other means of detection, such as other chromogenic, radioactive, chemiluminescent and fluorescent labeling, to simultaneously detect multiple targets, such a gene, a protein, and a chromosome, in a biological sample. More particularly the multiple targets may be labeled with the specifically deposited metal and other chromogenic labels to allow chromogenic immunohistochemical (IHC) detection in situ by using bright field light microscope.

### BACKGROUND OF THE INVENTION

Following the determination of the structure of DNA, it was not until 15 years later that methods for localizing DNA and RNA at the molecular level using complementary sequences as probes began to emerge. The development of the method was driven by the discovery that chemical denaturation procedures such as high temperature or sodium hydroxide treatment could alter the staining properties of different regions along chromosomes, leading to the development of banding profiles that could be used to identify and localize chromosomal features. De Jong H. (2003) Genome, 46:943-6. Differential staining with fluorescent dyes was used to elucidate banding structures within chromosomes. Caspersson et al. (1972) Int. Rev. Exp. Pathol 11:1-72. The genesis of modern in situ hybridization was the discovery that specific sequences localized to these bands. Initially, detection utilized radioisotopically labeled RNA probes to localize complementary DNA in tissue sections and cell spreads using autoradiography: in one of the three earliest reports, Pardue and Gall used Thymidine-³H-labeled RNA fractions extracted from cultured mouse and Xenopus cellular extracts to localize complementary DNA in denatured tissue sections. Pardue and Gall (1969) PNAS 64:600-4. Although cumbersome and limited in resolution by the scattering cone of radioactive emissions, these early experiments effectively demonstrated the localization of specific DNA fractions to chromosomal regions.

The technique of in situ hybridization achieved wider use as a number of enabling technologies were developed. Improved probe preparation and labeling methods, including random prime labeling, nick translation reaction, PCR-based labeling, and improved methods for cloning target sequences, made probe preparation more convenient and affordable. In particular, fluorescent labels were introduced to provide higher resolution and simpler visualization, and to avoid the hazards of radioactive probes and the long delay required to develop the autoradiographic signal. These enabled the technique of fluorescence in situ hybridization, or FISH. Trask (1991) Trends in Genetics 7:149-154. Labeling may be direct, in which the fluorescent label is linked directly to the nucleic acid hybridization probe, or indirect, in which the hybridization probe is bound in turn by a labeled secondary probe such as fluorescently labeled antibody or protein targeted to a unique feature or hapten incorporated into the hybridization probe. FISH has been used for mapping repetitive and single-copy DNA sequences on metaphase chromosomes, interphase nuclei, chromatin fibers, and naked DNA molecules, and for chromosome identification and karyotype analysis through the localization of large repeat families, typically the rDNAs and major tandem array families. However, despite the wide usage and the sensitivity of FISH in detection of many biomarkers, FISH is not always ideal for clinical diagnostic practice because of the requirement of complex equipment and the fluorescent signal degrades quickly. Thus, with the fast development of clinical diagnosis utilizing biomarkers coupled with therapeutic treatment, there is an increasing need for alternative methods for detection of biomarkers that require less complex equipment, enable faster throughput, and can provide more information for the practicing pathologist.

### SUMMARY OF THE INVENTION

The present invention provides innovative compositions, kits, assembles of articles and methodology for detecting multiple target molecules in a sample, such as in a tissue sample. In particular, site-specific deposition of elemental metal is used in conjunction with other means of detection, such as other chromogenic, radioactive, chemiluminescent and fluorescent labeling, to simultaneously detect multiple targets, such as a gene, a protein, and a chromosome, in a biological sample. More particularly, the multiple targets may be labeled with the specifically deposited metal and other chromogenic labels to allow chromogenic immunohistochemical (IHC) detection in situ by using bright field light microscope. The biological samples include, but are not limited to, cell cultures or mixtures, cytological specimens, tissue slices or sections, biopsies, and samples contains nuclei of cells, in a form of liquid, suspension, solid and immobilized to a solid support such as a slide.

In addition, site-specific and selective deposition of elemental metal in the targeted site or molecules not only facilitates chromogenic detection of the signals, but also allows cell morphology and in situ hybridization (ISH) signal to be viewed at the same time, and provides accurate results using standard equipment, such as bright field-microscopes. There is no fading of the sample upon observation or storage or exposure to room lights. Autofluorescence from cells and other molecules does not create any interference. Standard stains (such as nuclear fast red, hematoxylin and eosin) can be used and seen simultaneously with the enzyme deposited metals, making visualization of landmarks of a tissue simple.

In one aspect of the invention, a method is for detecting a plurality of target molecules in a test sample in vitro. The method comprises: detecting a first target molecule of the plurality of target molecules by i) binding an enzyme to the first target molecule, ii) contacting the enzyme with metal ions in the presence of an oxidizing agent and a reducing agent, whereby the metal ions are reduced to elemental metal, thereby depositing the elemental metal in the vicinity of the enzyme, and iii) determining the presence, amount or level of the deposited metal in the vicinity of the enzyme bound to the first target molecule; and detecting a second different target molecule of the plurality of target molecules in the test sample by generating a detectable signal at the site of the second target molecule that is different from the signal of the deposited metal.

As used herein “metal deposition” is defined as a buildup or accumulation of metal (metallic elements in the zero oxidation state) in the vicinity of the enzyme.. Typically, metal deposition will start within a distance of about 1 micron from the enzyme, but deposition may start 0.005, 0.01, 0.1, 5, 10, 50, 100, 1000 microns from the enzyme. Naturally as metal deposition continues the metal accumulation may extend beyond this distance.

In a preferred embodiment, the enzymatic metal deposition of the invention allows deposition of silver metal in the presence of peroxidase and activating agents with high sensitivity combined with high resolution and minimal background for in situ hybridization (ISH) detection, and visualization in the conventional bright field microscope without the need for oil immersion. Such an assay is herein termed as “Silver In Situ Hybridization” (SISH). In particular, the enzymatic metal deposition of the invention allows detection of a single copy of a target gene in a chromosome by a conventional bright field microscope without requiring oil immersion. The invention also enables detection of gene copies with a resolution that allows for individual enumeration of signals, such as discrete metal deposit dots for individual gene copies. In a variation of the embodiment, the invention allows for detection of at least 2, 3, 4, 5, 6, 7 or 8 copies of a target gene per nucleus, such as HER2 gene in human chromosome 17, as discrete metal deposit dots.

According to the method, the step of binding the enzyme to the first target molecule includes binding the enzyme to the first target molecule via a primary antibody that specifically binds to the first target molecule, and a secondary antibody that is conjugated with the enzyme and binds to the primary antibody.

Optionally, the step of binding includes binding the enzyme to the first target molecule via a nucleic acid probe that specifically hybridizes to the first target molecule and is labeled with detectable marker, wherein the enzyme binds to the detectable marker via an antibody that specifically binds to the detectable marker. Examples of the detectable marker include but are not limited to biotin, digoxingenin, dinitrophenyl (e.g., 2,4-dinitrophenyl (DNP), a radio-isotope or a fluorescent label such as fluorescein isothiocyanate (FTIC), Texas Red, rhodamine and Cy5. The enzyme may bind to the detectable marker via a primary antibody that specifically binds to the detectable marker, and a secondary antibody that is conjugated with the enzyme and binds to the primary antibody.

The target molecule may be a target gene, gene product, chromosome or genome. For example the target gene is a gene encoding an angiogenic growth factor receptor selected from the group consisting of receptor for fibrin (VE-cadherin), receptors for VEGF (Flt1 and KDR), receptor for VEGF-C and VEGF-D (Flt4), receptor for VEGF-165 (NP-1 and NP-2), receptors for angiopoeitin-1, -2, -3, and -4 (Tie1 and Tie2), receptors for FGF (FGF-R1, -R2, -R3 and -R4), receptor for PDGF (PDGF-R), receptor for ephrine A1-5 (Eph A1-8), and receptor for ephrine B1-5 (Eph B1-8). Optionally, the target gene is a gene encoding a receptor tyrosine kinase selected from the group consisting of epidermal growth factor receptors (EGFR), platelet-derived growth factor receptors (PDGFR), vascular endothelial growth factor receptors (VEGFR), nerve growth factor receptors (NGFR), fibroblast growth factor receptors (FGFR), insulin receptors, ephrin receptors, Met, and Ror. Examples of the epidermal growth factor receptor include HER1, HER2/neu (or HER-2/neu), HER3, or HER4. Also optionally, the target gene encoding a non-receptor tyrosine kinase selected from the group consisting of Kit (such as c-Kit), Src, Fes, JAK, Fak, Btk, Syk/ZAP-70, and Abl. The method further comprises: comparing the presence, amount or level of the deposited metal with that of a reference sample; and determining a disease status of a patient from whom the test sample is derived.

The reference sample may comprise a cell or tissue from a normal, healthy tissue, or from another disease tissue with known disease status, such as a breast tumor tissue.

The disease status may be disease determination or classification, prognosis, drug efficacy, patient responsiveness to therapy, whether adjuvant or combination therapy is recommended, or likelihood of recurrence of disease. Examples of the disease include but are not limited to benign tumors, cancer, hematological disorders, autoimmune diseases, inflammatory diseases, cardiovascular diseases, nerve degenerative diseases and diabetes.

Optionally, the disease status is patient responsive to therapy. For example, the disease is breast cancer; the therapy is trastruzumab or HERCEPTIN therapy; and the target molecule is HER2/neu gene or protein. For another example, the disease is gastrointestinal stromal tumor (GIST); the therapy is imatinib mesylate or GLEEVEC therapy; and the target molecule is c-Kit gene or protein.

According to the method, the weight ratio of the metal ions to the reducing agent may range from 1:5 to 5:1, and the weight ratio of the reducing agent to the oxidizing agent may range from 1:10 to 10:1.

Optionally, according to the method, the step of contacting includes: a) combining the enzyme with the metal ions; b) incubating the mixture of the enzyme and the metal ions at about 4-40° C. for about 1-10, 2-8 or 3-5 minutes; c) combining the incubated mixture of the enzyme and the metal ions with the reducing agent and the oxidizing agent; and d) incubating the mixture of the enzyme, the metal ions, the reducing agent and the oxidizing agent at about 4-40° C. for about 1-30, 2-20, 5-15, or 8-14 minutes.

Optionally, according to the method, the step of contacting includes a) combining the enzyme with the metal ions; b) incubating the mixture of the enzyme and the metal ions at about 4-40° C. for about 1-10, 2-8 or 3-5 minutes; c) adding the reducing agent to the mixture of step b); d) adding the oxidizing agent to the mixture of step c), and incubating at about 4-40° C. for about 1-30, 2-20, 5-15, or 8-14 minutes.

The method optionally further comprises: stopping the deposition of the elemental metal to the vicinity of the enzyme after a certain period of time. The step of stopping may include washing away residual metal ions from the enzyme. Optionally, the step of stopping includes rinsing the enzyme with a solution selected from the group consisting of a solution combining sodium thiosulfate and ammonium chloride, a solution of potassium thiocyanate, a solution combining potassium ferricyanide and sodium thiosulfate, a solution of potassium ferricyanide, a solution of sodium thiosulfate, and a solution of sodium periodate.

The method optionally further comprises: detecting the elemental metal deposited in the vicinity of the enzyme by automatallography or by bright field light microscopy.

When coupled with other means of detection, such as another chromogenic (e.g., 3,3′-diaminobenzidine (DAB), 3-Amino-9-ethylcarbazole (AEC), Bajoran Purple, Fast Red and Ferangi Blue), colorimetric, radioactive, fluorescent and chemiluminescent labels, multiple targets in a biological sample can be simultaneously detected.

By coupling site-specific deposition of metal with additional detection methods including by x-rays, electron microscopy, electrochemical, optical, magnetic detection, more sensitive and/or rapid detection of targets in a biological sample is possible compared with conventional test methods.

In particular, the present invention can be used for detection of multiple biomarkers for research, diagnosis, prevention and treatment of diseases and conditions. For example, the inventive method can be used to determine a disease status of a mammal, preferably a human subject, by detecting the levels of the multiple biomarkers in a sample derived from the mammal. Such “disease status” may relate to disease determination or classification, prognosis, drug efficacy, patient responsiveness to therapy (the so-called “targeted therapy”), whether adjuvant or combination therapy is recommended, likelihood of recurrence of disease, or the like.

The present invention also provides a kit for detecting multiple targets in a sample, comprising: metal ions selected from the group consisting of silver, gold, iron, mercury, nickel, copper, platinum, palladium, cobalt, iridium ions and a mixture thereof; an oxidizing agent; a reducing agent; and an enzyme. Preferably, the weight ratio of the metal ions to the reducing agent ranges from 1:5 to 5:1, and the weight ratio of the reducing agent to the oxidizing agent ranges from 1:10 to 10:1.

The kit may further comprise a plurality of binding moieties or agents each of which binds to a different target molecule, such as an antibody, antibody fragments, peptide, nucleic acids, nucleic acid probes, carbohydrates, drugs, steroids, products from plants, animals, humans and bacteria, and synthetic molecules. For example, the target is a target gene or genome and the binding moiety is a nucleic acid probe that binds to the target gene or genome. The enzyme may bind to the target via a primary antibody that binds to the target, or via a primary antibody that binds to the binding moiety. Optionally, the enzyme is conjugated to a secondary antibody that binds to the primary antibody. Preferably, the enzyme is peroxidase; the metal ions are silver ion; the oxidizing agent is hydrogen peroxide; and the reducing agent is hydroquinone.

The kit may further comprise instruction for performing a process of depositing elemental metal in the vicinity of the enzyme.

According to the present invention, examples of the metal ions include but are not limited to silver, gold, iron, mercury, nickel, copper, platinum, palladium, cobalt, iridium ions and a mixture thereof. Preferably, the metal ions are silver ions.

Examples of the enzyme include but are not limited to oxido-reductases (such as dehydrogenases, oxidases); hydrolases (such as esterases, lipases, phosphatases, nucleases, carbohydrases, proteases); transferases; phosphorylases; decarboxylases; hydrases; and isomerases. Preferably, the enzyme is peroxidase. More preferably, the enzyme is horseradish peroxidase. The peroxidase can utilize a colorless organic substrate capable of being converted by the peroxidase to a colored substrate. Examples of the colorless organic substrate is 3,3′-diaminobenzidine or 5-bromo-4-chloro-3-indolyl phosphate. The enzyme may be optionally conjugated to streptavidin, or to an antibody.

The oxidizing agent may be an oxygen-containing oxidizing agent, such as hydrogen peroxide.

Examples of the reducing agent include but are not limited hydroquinone, a hydroquinone derivative, n-propyl gallate, 4-methylaminophenol sulfate, 1,4 phenylenediamine, o-phenylenediamine, chloroquinone, bromoquinone, 2-methoxyhydroquinone, hydrazine, 1-phenyl-3-pyrazolidinone and dithionite salts.

According to the present invention, preferably the enzyme is a peroxidase; the metal ions are in a form of silver acetate; the oxidizing agent is hydrogen peroxide; and the reducing agent is hydroquinone. The weight ratio of silver acetate to hydroquinone ranges from about 1:2 to about 4:1, optionally from about 1:1 to about 3:1, or optionally from about 1:1 to about 2:1. The weight ratio of hydroquinone to hydrogen peroxide ranges from 1:2 to 6:1, optionally from about 1:1 to about 4:1, or from about 1:1 to about 3:1, or optionally about 1:1 to about 2:1.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graphical representation of one embodiment of the present invention. The figure represents detection of three target molecules using label conjugated and/or enzyme-conjugated primary and secondary antibodies.

FIG. 2 is a photomicrograph at 100× showing dots of deposited silver metal as discrete single copies of HER2 gene in a 4 micron-section of normal breast duct tissue.

FIG. 3 is a photomicrograph at 100× showing dots of deposited silver metal as discrete single copies of HER2 gene in a 4 micron-section of breast tumor tissue that is not amplified for HER2.

FIG. 4 is a photomicrograph at 100× showing dots of deposited silver metal as numerous copies of HER2 gene in a 4 micron-section of breast tumor tissue with low levels of HER2 gene amplification, from 3 to 5 copies of HER2 gene present in the tumor cells.

FIG. 5 is a photomicrograph at 100× showing clusters of deposited silver metal as many copies of HER2 gene in a 4 micron-section of breast tumor tissue with relatively higher levels of HER2 gene amplification (than that of breast tumor tissue shown in FIG. 4), with 6 or greater copies of HER2 gene present in the tumor cells.

FIG. 6 is a photomicrograph at 100× showing clusters of deposited silver metal as many copies of HER2 gene in a 4 micron-section of breast tumor tissue with even higher levels of HER2 gene amplification (than that of breast tumor tissue shown in FIG. 5).

FIG. 7 is a photomicrograph showing dots of deposited silver metal as discrete single copies of HER2 gene in a breast tumor tissue that is not amplified for HER2.

FIG. 8 is a photomicrograph showing clusters of deposited silver metal as many copies of HER2 gene in a breast tumor tissue with relatively higher levels of HER2 gene amplification (than that of breast tumor tissue shown in FIG. 4), with 6 or greater copies of HER2 gene present in the tumor cells.

FIG. 9 is a graphic depiction of the deposition of metal in the vicinity of a target molecule. In this depiction, a DNA probe labeled with a hapten is bound to a target sequence. The hapten (represented by the filled triangle) is bound by an anti-hapten primary antibody. The primary antibody is bound by a horseradish peroxidase-conjugated secondary antibody. Metal ions in solution are reduced to elemental metal in the vicinity of the bound enzyme.

FIG. 10 is a graphic depiction of the metal deposition used in combination with other signals. Panel A shows a surface protein bound by an alkaline-phosphatase-conjugated antibody which produces a colorimetric signal upon addition of an appropriate substrate. Panel B shows a DNA probe labeled with a fluorescent marker hybridized to a target sequence. Panel C shows a DNA probe labeled with a hapten bound to a target sequence. The hapten is bound by an anti-hapten primary antibody. The primary antibody is bound by an antibody with multiple horseradish peroxidase enzymes conjugated to it, which leads to the deposition of metal in the vicinity of the bound antibody. Panel D depicts a cell to which the three different probes are bound. Because of the localization of each signal, concurrent detection of all three target molecules is possible.

FIG. 11 is a photomicrograph at 100× showing dots of deposited silver metal as discrete single copies of HER2 gene in a 5 μm-section of formalin-fixed, paraffin-embedded MCF7 (a “highly rearranged, near triploid” human breast carcinoma cell line) xenograft tumor. There are one to three signals per cell, dependent on the cell cycle stage and how each cell was cut.

FIG. 12 is a photomicrograph at 100× showing dots of deposited silver metal as discrete single copies of HER2 gene in a 5 μm-section of formalin-fixed, paraffin-embedded ZR-75-1 (A highly rearranged, near triploid human breast carcinoma cell line) xenograft tumor.

FIG. 13 is a photomicrograph at 100× showing clusters of dots of deposited silver metal as clusters of HER2 gene in a 5 μm-section of formalin-fixed, paraffin-embedded BT-474 (human breast ductal carcinoma cell line) xenograft tumor. As can readily be seen, there are amplified HER2 gene signals in each cell.

FIG. 14 is a photomicrograph at 100× showing dots of deposited silver metal as discrete single copies of HER2 gene in a 5 μm-section of MCF7 xenograft tumor. Also shown are red dots resulting from fast red naphthol staining indicating single copies of chromosome 17 centromere (CEP 17).

FIG. 15 is a photomicrograph at 100× showing dots of deposited silver metal as discrete single copies of HER2 gene in a 5 μm-section of ZR-75-1 xenograft tumor. Also shown are red dots resulting from fast red naphthol staining indicating single copies of CEP 17.

FIG. 16 is a photomicrograph at 100× showing dots of deposited silver metal as clusters of HER2 genes (due to amplification in copy number) in a 5 μm-section of BT-474 (human breast ductal carcinoma) xenograft tumor. Also shown are red dots resulting from fast red naphthol staining indicating single copies of CEP 17.

FIG. 17 is a photomicrograph at 100× showing dots of deposited silver metal as discrete single copies of HER2 gene in a 5 μm-section of MCF7 xenograft tumor. Also shown are red dots resulting from fast red naphthol staining indicating single copies of CEP 17.

FIG. 18 is a photomicrograph at 100× showing dots of deposited silver metal as discrete single copies of HER2 gene in a 5 μm-section of ZR-75-1 xenograft tumor. Also shown are red dots resulting from fast red naphthol staining indicating single copies of CEP 17.

FIG. 19 is a photomicrograph at 100× showing clusters of deposited silver metal as clusters of HER2 genes (due to amplification in copy number) in a 5 μm-section of BT-474 (human breast ductal carcinoma) xenograft tumor. Also shown are red dots resulting from fast red naphthol staining indicating single copies of CEP 17.

FIG. 20 is a graphic depiction of the triple-staining protocol used to detect HER2 gene, CEP 17 and HER2 protein as described in Example 5.

FIG. 21 is a photomicrograph at 100× showing dots of deposited silver metal as discrete single copies of HER2 gene in a 5 μm-section of MCF7 xenograft tumor. Also shown are red dots resulting from fast red naphthol staining indicating single copies of CEP 17. The sample has also been subjected to immunohistochemical (IHC) staining for HER2 protein.

FIG. 22 is a photomicrograph at 100× showing dots of deposited silver metal as discrete single copies of HER2 gene in a 5 μm-section of ZR-75-1 xenograft tumor. Also shown are red dots resulting from fast red naphthol staining indicating single copies of CEP 17. Immunohistochemical staining of HER2 protein demonstrates the presence of the protein.

FIG. 23 is a photomicrograph at 100× showing dots of deposited silver metal as clusters of HER2 genes (due to amplification in copy number) in a 5 μm-section of BT-474 (human breast ductal carcinoma) xenograft tumor. Also shown are red dots resulting from fast red naphthol staining indicating single copies of CEP 17. Immunohistochemical staining of HER2 protein demonstrates the presence of the protein.

### DETAILED DESCRIPTION OF THE INVENTION

The present invention provides innovative compositions, kits, assembles of articles and methodology for detecting multiple target molecules in a sample, such as in a tissue sample. In particular, site-specific deposition of elemental metal is used in conjunction with other means of detection, such as other chromogenic, radioactive, chemiluminescent and fluorescent labeling, to simultaneously and sensitively detect multiple targets, such as a gene, a protein, and a chromosome, in a biological sample. Such site-specific and selective deposition of elemental metal in the targeted site or molecules not only facilitates chromogenic detection of the signals, but also allows cell morphology and in situ hybridization (ISH) signal to be viewed at the same time, and provides accurate results using standard equipment, such as bright field-microscopes.

### 1. Site Directed Deposition of Elemental Metal

One aspect of the invention is to utilize enzyme-catalyzed deposition of elemental metal in the vicinity of a target in a sample. This methodology is described in detail in U.S. Pat. Nos. 6,670,113 and 7,183,072, and in U.S. patent application Ser. No. 11/714,682, filed on Mar. 5, 2007, which are incorporated herein by reference in their entirety.

It has been found that enzymes can accept metal ions themselves as a substrate and reduce those metal ions to metal. Further the enzymes can deposit the reduced metal. For example, if horseradish peroxidase is combined with silver ions (silver acetate was used originally), and an appropriate reducing agent is added, e.g., hydroquinone, no enzyme-mediated reduction of metal occurs. However, upon addition of hydrogen peroxide, the enzyme accepts silver ions as a substrate and reduces them to silver metal, resulting in a metallic deposit.

Pretreatment of the enzyme with gold ions (e.g., from potassium tetrabromoaurate), or silver ions (e.g., from silver acetate), followed by optional washing (to remove the excess pretreatment metal ion solution), results in greatly enhanced rates of silver deposition when the developing mix was subsequently applied. As used herein the term “developing mix” is defined as the solution applied to the enzyme to obtain metal deposition. Typically the developing mix contains metal ions (e.g., silver acetate), a reducing agent (e.g., hydroquinone) and an oxidizing agent (e.g., hydrogen peroxide) in a controlled pH buffer (e.g., 0.1M sodium citrate, pH 3.8). In the above enzymatic metal reduction the developing mix advantageously comprised silver acetate, hydroquinone, and hydrogen peroxide in a citrate buffer at a pH of about 3.8. The enzymatic metal reduction and deposition can be conveniently observed when the enzyme is immobilized, for example, either on nitrocellulose paper, or immunologically attached to a target antigen.

According to present invention, any suitable silver ions, such as silver acetate, silver lactate and silver nitrate, can be used in the peroxidase catalyzed silver deposition reactions. Other metal ions solutions, such as solutions of mercurous chloride, cesium chloride, lead nitrate, nickel sulfate, copper sulfate, palladium acetate and potassium ferrocyanide, may also be used

Other enzymes are also active toward reducing metal ions from their salts. For example, with a pretreatment of potassium tetrabromoaurate, catalase was found to reduce silver ions to silver metal when hydroquinone and hydrogen peroxide were included in a sodium citrate buffer at pH 3.8. Additionally lactoperoxidase was found to be active with silver ions.

While hydroquinone is presently the best known reducing agent, other reducing agents are also believed to be useful in practicing the invention, including, for example, n-propylygallate, 4-methylaminophenol sulfate, 1,4 phenylenediamine, o-phenylenediamine, chloroquinone, bromoquinone, 2-methoxyhydroquinone, hydrazine, metol, ascorbic acid, 1-phenyl-3-pyrazolidinone (phenidone aminophenol) and dithionite salts such as sodium dithionite. 1-phenyl-3-pyrazolidinone, sodium borohydride and boranes may work as a reducing agent without the need for H₂O₂.

Enzymes may be coupled one after another to produce the desired metal deposit. For example, glucose serves as a substrate for glucose oxidase, producing hydrogen peroxide. The hydrogen peroxide then serves as a substrate for peroxidase to deposit silver ions in the presence of hydroquinone, because hydrogen peroxide is used in that enzyme reaction.

The enzyme altered metal product may be soluble or dispersible in water. Naturally in other embodiments of the invention the altered metal products can be soluble in organic solvents. For example, aurothioglucose with hydroquinone and hydrogen peroxide, when exposed.to horseradish peroxidase bound to nitrocellulose, turns an intense yellow at the location of the enzyme, if left undisturbed. However, rinsing or agitation easily disperses the color. This reaction may be coupled to an optical density reader or used in an ELISA format with a microtiter plate reader that will sense the newly formed colored product.

The metal deposits, formed from particles or ions, may be further intensified using autometallography. As used herein “autometallography” is defined as a deposition of metal from metal ions in solution that specifically occurs on a nucleating metal surface. For example, it is known that if gold particles in the size range of about 1 to 50 nm are exposed to silver ions and a reducing agent, silver metal is deposited on the gold particles forming a composite particle. As more silver is deposited the composite particle increases in size. As the composite particles become larger, they become more visible and detectable.

Autometallography may be combined with the enzymatic metal deposition. Once metal has been enzymatically deposited as a metal particle, the metal particle is subjected to an autometallographic solution. The autometallographic deposit forms a composite particle and amplifies the size of the enzymatically deposited metal particle so that the enzymatic metal deposits are more voluminous and hence easier to detect. The combination of enzymatic metal deposition in tandem with autometallography provides increased sensitivity and/or more rapid detection.

A further advantage of the use of enzymatic metal deposition in tandem with autometallography is that the autometallographic deposit may be a different metal from that of the enzymatically deposited particle. This permits overcoating of the original enzymatic metal deposit by one or more different autometallographically deposited metal layers. The autometallographic layer may also become the bulk of the composite particle if desired. It should be noted that the autometallographic coating, or coatings, may confer new properties, to the enzymatic metal deposit, such as altered oxidation rates, magnetic properties, optical properties and electrical properties. For example, if gold is autometallographically deposited over an enzymatic silver deposit, this would confer improved chemical resistance as gold is more noble and more resistant to oxidation than the core silver deposit. Autometallographically depositing copper over a enzymatic metal deposit would confer the conductive, and other, properties of copper to the metal particle.

A wide variety of methods can be used to detect and observe the enzymatic metal deposits. Some of the methods useful for detecting and observing an enzymatic metal deposit include:

Visual observation using the unaided eye. The enzymatic deposition of silver or silver after pretreating with gold typically results in a black or brown color due to the presence of finely divided metal. The color is easily seen by the unaided eye, especially against a light colored background.

Visual observation using a microscope. For higher resolution or more sensitive detection, the metal deposit may be viewed under a microscope. The metal is very dense and opaque, and may in many circumstances be easily detected by this density using bright field illumination. This feature is useful in chromogenic in situ hybridization (CISH) assays for detecting a target site or molecule in a sample. Compared with fluorescence in situ hybridization (FISH), CISH is a technique that allows in situ hybridization methods to be performed and detected with a bright field microscope, instead of a fluorescence microscope as required for FISH. While FISH requires a modern and expensive fluorescence microscope equipped with high-quality 60× or 100× oil immersion objectives and multi-band-pass fluorescence filters which are not used in most routine diagnostic laboratories, CISH allows detection with standard light (bright field) microscopes which are generally used in diagnostic laboratories. Also, with FISH, the fluorescence signals can fade within several weeks, and the hybridization results are typically recorded with an expensive CCD camera, while the results of CISH do not generally fade allowing the tissue samples to be archived and reviewed later. Therefore, analysis and recording of FISH data is expensive and time consuming. Most importantly, tissue section morphology is not optimal in FISH. Generally, histological detail is better appreciated with bright-field detection, which is possible with CISH detection. A further advantage of CISH is that large regions of the tissue section can be scanned rapidly after CISH counterstaining because morphological detail is readily apparent using low power objectives (e.g. 100× and 20×), while FISH detection generally requires substantially higher magnification, thus reducing the field of view. Such features like efficient processing and convenient viewing allow high throughput, automatic screening of a large number of samples by using automated sample processing and detection devices, such as automated immunohistochemistry slide preparation and staining devices. A typical example of such automated devices is the Benchmark™ XT automated platform provided by Ventana Medical Systems, Tucson, Ariz. Details of the Benchmark™ XT automated platform are described in the manufacturer's user manual, product description and alike, and in U.S. Pat. No. 6,296,809, which are herein incorporated by reference in their entirety.

Reflectance. Because metals reflect light, epi-illumination may be used either with or without a microscope. Additionally, metals repolarize light upon reflection, so crossed polarizers may be used to filter out reflections from non-metallic material, thus improving the signal-to-noise ratio.

Electron microscopy. Metals are clearly seen via electron microscopy due to their density in transmission electron microscopy. They also have high backscatter coefficients, and may be viewed with a backscatter detector on a scanning electron microscope. Metals also give off characteristic x-rays upon electron bombardment, so they may be detected by x-ray detectors, or electron energy loss spectrometers. Other methods include detection of the characteristic electron diffraction patterns of metals.

Polarographic, electrochemical, or electrical detection. Metals deposited on an electrode alter its properties. By probing with the proper currents and voltages, metal can be detected.

X-ray spectroscopy. Metals can be detected by x-ray induced fluorescence or x-ray absorption.

Chemical tests. Sensitive tests exist for chemically converting metals into products that are colored or otherwise detectable.

Mass detection. The mass of the deposited metal is detected using, for example, a quartz crystal mass balance. A quartz crystal in an inductance-resistance-capacitance (LRC) electronic circuit with an alternating voltage supply oscillates at some resonant frequency. If metal is deposited on the surface of the quartz crystal, this changes the mass of the crystal and its resonant frequency. This provides a very sensitive method for measuring mass changes.

Light scattering. Fine metal deposits will alter the light scattering of a solution or surface.

Other optical methods of detecting interaction of metals with light, including absorption, polarization and fluorescence, may be utilized.

Magnetic detection. The magnetic properties of the deposited metal can be detected using appropriate equipment such as magnets, coils, or sensing optical-magnetic property changes.

Autometallography. Further amplification of the signal may be achieved by applying additional metal ions and reducing agent and other additives to effect further metal deposition that specifically nucleates on the initial enzymatic metal deposit.

Scanning probe microscopy. Metal deposits may be recognized at high spatial resolution and sensitivity by the various scanning probe microscope techniques, including scanning tunneling microscopy (STM), atomic force microscopy (AFM), near field optical microscopy (NSOM) and other related techniques using piezoelectrically driven scanned tips.

These reagents for carrying out the enzymatic metal deposition can be assembled into kits. As used herein, a “kit” refers to any delivery system for delivering materials or reagents for carrying out a method of the invention. In the context of reaction assays, such delivery systems include systems that allow for the storage, transport, or delivery of reaction reagents (e.g., oxidizing agents, reducing agents, metal ion solutions, probes, enzymes, etc. in the appropriate containers) and/or supporting materials (e.g., buffers, written instructions for performing the assay and trouble shooting, etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

The enzymatic metal deposits are useful in the present invention for detecting multiple targets in a biological sample, including but not limited to:

Immunohistochemistry and Immunocytochemistry:

Currently, primary antibodies are widely used to target antigens on cytologic specimens, tissue sections, and biopsies. The antibodies are then commonly detected by a variety of techniques including use of a secondary antibody that is biotinylated, followed by avidin-biotin-peroxidase complex (ABC complex), and development of a brown color with 3,3′-diaminobenzidine (DAB). Optionally coupled with other detection methods include fluorescence and chemiluminescence, the metal deposition method may be used as a detection scheme by supplying metal ions and a developing mix, preferably with a metal ion pretreatment, to the peroxidase localized to the antigen by the above described or analogous methods. Instead of depositing DAB, a metal, for example silver will be deposited. Enzymatic silver deposition has the advantage of better detectability not only by bright field microscopy (where a black deposit is formed), but also by reflectance microscopy, electron microscopy and other methods suited to metal detection. Sensitivity is therefore greatly improved over conventional methods.

Alternatively, the metal clusters or colloids with surface enzyme substrates described above may be used to form antigen-specific deposits by enzymatic action. The enzymatic deposition of metals has a great advantage over simple targeting of metal nanoparticles attached to antibodies, as is commonly done using gold-antibody conjugates for electron microscopy, lateral flow tests, and other applications. One major advantage is that the metal or metal particles are continuously deposited by the enzyme, as long as more substrate is provided. In this way, huge amounts of metal product may be deposited compared to non-enzymatic targeting of metal particles, achieving a desirable amplification effect.

In situ Hybridization

Presently considerable laboratory and pathological testing is being done and it is desirable to have a sensitive method to detect DNA or RNA sequences in situ, either as part of an intact genome or segments thereof. By hybridizing a complementary probe associated with a detectable moiety to a targeted sequence, these sequences can be found and quantified. However the limits of detection required to see single gene copies or low levels of expression exceed the capability of many methods. The present invention may be used to achieve sensitive detection of multiple targets in a chromosome, single copies or low-level amplification of a target gene in a sample by having the nucleic acid probe labeled with an enzyme, for example peroxidase, or to use multistep labeling, such as a biotinylated probe, followed by avidin-biotin-peroxidase complex or biotin-antibody-peroxidase complex. The peroxidase is then utilized as previously described to deposit metals. The enzymatically deposited metals are highly detectable and as further described below reliable single gene sensitivity has been achieved using this method.

Lateral Flow, Blots, and Membrane Probing

The metal deposition method can be used in a number of useful applications, such as lateral flow diagnostics (e.g., the “dipstick” pregnancy test kit), Western, Southern, and other blots, and other tests performed on membranes, optionally in conjunction with other detection schemes such as radioactive, fluorescent, colloidal gold, chemiluminescent, calorimetric, and other detection schemes. For example, the target can be probed with a binding moiety that is associated with an enzyme, for example peroxidase. The binding moieties useful depend on the desired target and include, for example, antibody, antibody fragments, antigen, peptide, nucleic acids, nucleic acid probes, carbohydrates, drugs, steroids, natural products from plants and bacteria and synthetic molecules that have an affinity for binding particular targets. Enzymatic metal deposition is then applied, using either metal particles with substrate shells or metal ions and an appropriate developing mix, to deposit metal in the vicinity of the specific target site. The metal deposit may be in the form of an attached deposit or dispersed in solution. The enzymatically deposited metals are highly detectable and provide an extremely sensitive detection method.

Sensitive Detection of Antigens and Other Materials

Many other formats for detection of antigens and other materials have been devised, such as use of gels, cell cultures, tissue slices, microtiter plate systems and surface sensors. Most of these may easily be adapted to accommodate the present invention and substitute enzymatic deposition of metals and subsequent detection in place of conventional techniques. This would transform the conventional formats into new and improved formats having desirable characteristics such as higher sensitivity, lower cost, permanency of record and other advantages. Substitution of enzymatic deposition of metals and subsequent detection in place of conventional techniques also eliminates many of the disadvantages of conventional systems, such as use of radioactive materials, bleaching, transitory products and high expense.

Electron Microscope Probes

Small amounts of metals are easily detected in electron microscopes by their density, backscatter, x-ray emission or energy loss. The invention herein can be used to specifically target antigens or other sites, initially with an enzyme followed by deposition of metal. The enzymatically deposited detectable metal allows the targeted sites to be analyzed with high specificity and sensitivity.

### 2. Examples of Target Molecules in Biological Samples

The present invention can be applied to detection of a wide variety of biological molecules for research, genetic profiling, diagnosis, prevention and/or treatment of diseases and conditions. For example, the inventive method can be used to determine a disease status of a mammal, preferably a human subject, by detecting the level of the biomarker in a sample derived from the mammal. Such “disease status” may relate to disease determination or classification, prognosis, drug efficacy, patient responsiveness to therapy (the so-called “targeted therapy”), whether adjuvant or combination therapy is recommended, likelihood of recurrence of disease, or the like. For example, information on changes in levels of biomarkers in patients in response to drugs in clinical trials or treatment can be utilized to stratify patients into sub-populations that are more or less responsive to a particular drug, or susceptible to adverse side effects of the drug; a higher amount of the target biomarker in a patient sample in comparison with a reference sample of normal cells may indicate that the patient has a disease associated with aberrant amplification and/or expression of the biomarker.

Accordingly, the present invention provides a method for detecting a plurality of target molecules such as biomarkers in a test sample, preferably in vitro, comprising: detecting a first target molecule of the plurality of target molecules by i) binding an enzyme to the first target molecule, ii) contacting the enzyme with metal ions in the presence of an oxidizing agent and a reducing agent, whereby the metal ions are reduced to elemental metal, thereby depositing the elemental metal in the vicinity of the enzyme, and iii) determining the presence, amount or level of the deposited metal in the vicinity of the enzyme bound to the first target molecule; and detecting a second different target molecule of the plurality of target molecules in the test sample by generating a detectable signal at the site of the second target molecule that is different from the signal of the deposited metal.

For example, the presence, amounts or levels of the deposited metal and/or the detectable signal generated by the second target molecule may be compared with those of a reference sample so as to determine the difference in the profile of the target molecules in the test sample and the reference sample. Such information can be used to determine the disease status of the patient from whom the test sample is derived.

As used herein, the reference samples typically have one or more cell, xenograft, or tissue samples that are representative of a normal or non-diseased state to which measurements on patient samples are compared to determine whether a biomarker is present in excess or is present in reduced amount in the patient sample. The nature of the reference sample is a matter of design choice for a particular assay and may be derived or determined from normal tissue of the patient him- or herself, or from tissues from a population of healthy individuals. Preferably, values relating to amounts of the biomarker in reference samples are obtained under essentially identical experimental conditions as corresponding values for patient samples being tested. Reference samples may be from the same kind of tissue as that of the patient sample, or it may be from different tissue types, and the population from which reference samples are obtained may be selected for characteristics that match those of the patient, such as age, sex, race, and the like. In application of the invention, amounts of the biomarker on patient samples are compared to corresponding values of reference samples that have been previously tabulated and are provided as measured values, average ranges, average values with standard deviations, or like representations.

In one aspect, the method provided in the present invention can be used to detect receptors for angiogenic growth factors which belong to the family of the receptor tyrosine kinase and are intimately involved in tumor development and metastasis. Examples of such angiogenic growth factor receptors include, but are not limited to, receptor for fibrin (VE-cadherin), receptors for VEGF (Flt1 and KDR), receptor for VEGF-C and VEGF-D (Flt4), receptor for VEGF-165 (NP-1 and NP-2), receptors for angiopoeitin-1, -2, -3, and -4 (Tie1 and Tie 2), receptors for FGF (FGF-R1, -R2, -R3 and -R4), receptor for PDGF (PDGF-R), receptor for ephrine A1-5 (Eph A1-8), and receptor for ephrine B1-5 (Eph B1-8). Sensitive detection of such receptors allows early diagnosis, prognosis, or staging of tumors, benign, malignant, or metastatic, and other conditions associated abnormal angiogenesis or neovascularization.

The target molecules suitable for detection using the inventive method also include G protein coupled receptors (GPCR) such as receptor for sphingosie-1-phosphate or SPP and for lysophosphatidic acid or LSA (edg receptor), cytokine receptors such as receptor for tumor necrosis factor-α or TNF-α (TNF-α receptor) and receptor for interleukin-8 or IL-8 (IL-8 receptor), protease receptors such as receptor for urokinase (urokinase receptor), and integrins such as receptor for thromospondin-1 and -2 (αvβ3 integrin and α2vβ1 integrin) and receptor for fibronectin (αvβ3 integrin), and matrix metalloprotease. Also included are receptors for protein factors that have anti-angiogenic effects, such as receptor for angiostatin (angiostatin-R, also called Annexin II), receptor for angiostadin (angiostadin binding protein I), low-affinity receptors for glypicans, receptor for endostatin (endostatin-R), the receptor for endothelin-1 (endothelin-A receptor), receptor for angiocidin (angiocidin-R), the receptor angiogenin (angiogenin-R), receptors for thromospondin-1 and thromospondin-2 (CD36 and CD47), and the receptor for tumstatin (tumstatin-R). Still other target molecules include T-cell markers (e.g., CD3, CD4, CD8, TCR), B-cell markers (e.g. CD20), cell proliferation indicators (e.g., Ki-67), apoptosis related indicators (e.g. Caspase-3, CK8/18, p63), and lineage-specific markers/tumor cell indicators (e.g., CALB2, CD5, CD10, CD31, CD34, CDX2, CHGA, CK5, CK7, CK17, CK20, HSA, MART-1, Pax-5, PSA, S-100, SYP).

In another aspect, the present invention can be applied to detect a kinase gene or product thereof for diagnostic or therapeutic purposes. For example, the level of the gene or its product can be detected to aid in the assessment of patients who have diseases associated with abnormal activity of the kinase and could benefit from a therapy using an inhibitor of the kinase.

In one variation, the kinase is a serine/threonine kinase such as a Raf kinase; and the kinase inhibitor is BAY 43-9006.

In another variation, the kinase is a protein kinase kinase such as Raf-mitogen-activated protein kinase kinase (MEK) and protein kinase B (Akt) kinase.

In yet another variation, the kinase is an extracellular signal-regulated kinase (ERK). Examples of the inhibitor of ERK include but are not limited to PD98059, PD184352, and U0126.

In yet another variation, the kinase is a phosphatidylinositol 3′-kinase (PI3K). Examples of the inhibitor of PI3K include but are not limited to LY294002.

Examples of the receptor tyrosine kinase include, but are not limited to, epidermal growth factor receptor family (EGFR), platelet-derived growth factor receptor (PDGFR) family, vascular endothelial growth factor receptor (VEGFR) family, nerve growth factor receptor (NGFR) family, fibroblast growth factor receptor family (FGFR) insulin receptor family, ephrin receptor family, Met family, and Ror family.

Examples of the epidermal growth factor receptor family include, but are not limited to, HER1, HER2/neu (or HER2/neu), HER3, and HER4.

Examples of the inhibitors of epidermal growth factor receptor family include, but are not limited to, trastruzumab (HERCEPTIN®), ZD1839 (IRESSA®), PD168393, CI1033, IMC-C225, EKB-569, and inhibitors binding covalently to Cys residues of the receptor tyrosine kinase.

In particular, the transmembrane tyrosine kinase receptor HER2/neu was identified as an oncogene overexpressed by about 30% of breast cancers. These HER2/neu-overexpressing breast cancers define a subset of breast tumors that are characteristically more aggressive, and women who develop them have a shorter survival. Additionally, amplification of the HER2/neu gene correlates with higher proliferation and resistance to chemotherapy. Trastuzumab, a humanized monoclonal antibody specific for HER2/neu, has been widely used in the management of metastatic HER2/neu-overexpressing breast cancers. As a single agent, it produces response rates similar to those of many single-agent chemotherapeutic agents active in metastatic breast cancer and has limited toxicity. Combining trastuzumab with chemotherapy can result in synergistic antitumor activity. However, in the absence of HER2 overexpression, such treatment with trastuzumab is less effective and trastuzumab can cause cardiotoxicity and bleeding in combination. Thus, determining the level of expression, the level of amplification and/or the combination of the two is important in determining prognosis and treatment.

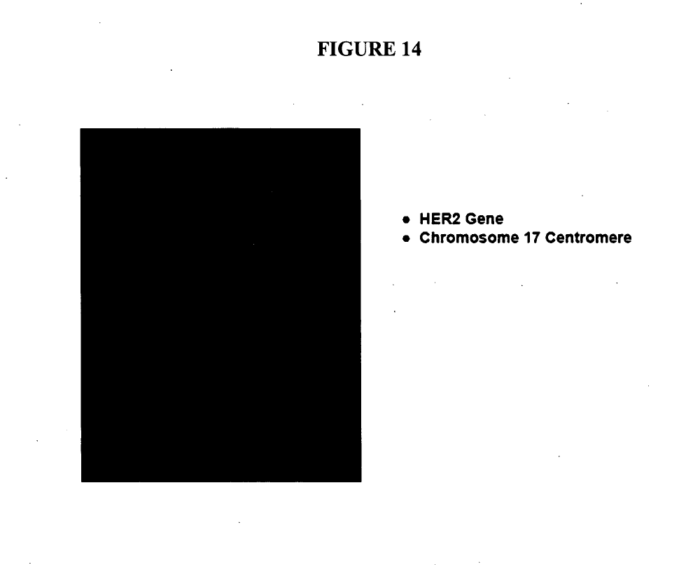

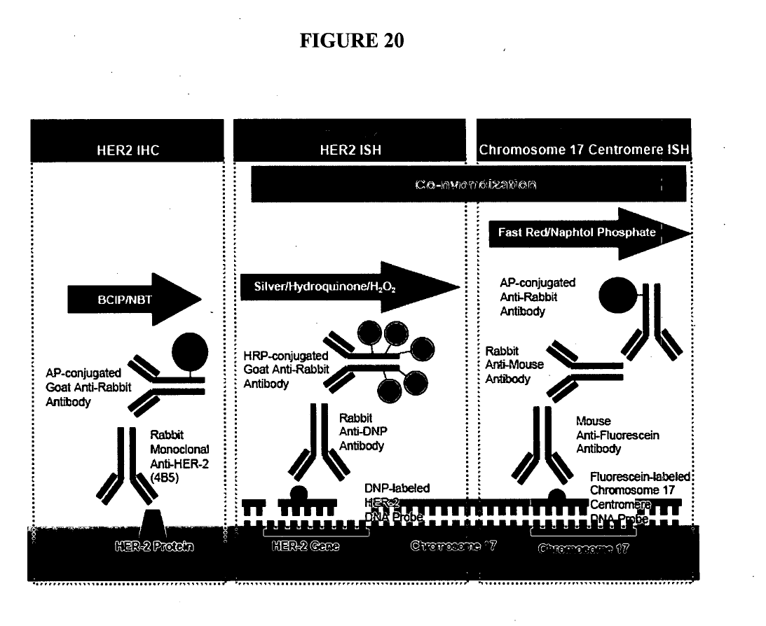

Thus, the method of the present invention can be used to detect the HER2 gene and/or its product to guide the selection of the patients who may be more responsive to therapeutic intervention targeting HER2 protein, such as a therapy of trastuzumab. For example, the present invention can be applied to detect HER2 gene amplification by using a nucleotide probe specific to HER2 gene in formalin-fixed, paraffin-embedded breast cancer tissue samples (See FIGS. 14-19). Additionally, the present invention can be applied to detect HER2 gene amplification and HER2 protein levels (See FIGS. 20-23). Both approaches aid in the assessment of breast cancer patients for whom treatment with trastuzumab is considered.

Examples of the vascular endothelial growth factor receptor family include, but are not limited to, VEGFR1, VEGFR2, and VEGFR3. An example of the inhibitor of the vascular endothelial growth factor receptor family includes, but is not limited to, SU6668.

Examples of the nerve growth factor receptor family include, but are not limited to, trk, trkb and trkC. Examples of the inhibitors of the nerve growth factor receptor family include, but are not limited to, CEP-701, CEP-751, and indocarbazole compound. Examples of the. diseases associated with abnormal activity of the nerve growth factor receptor family include, but are not limited to, prostate, colon, papillary and thyroid cancers, neuromas and osteoblastomas.

Examples of the Met family include, but are not limited to, Met, TPR-Met, Ron, c-Sea, and v-Sea. Examples of disease associated with activity of the receptor tyrosine kinase from Met family include, but are not limited to, invasively in-growing tumor, carcinoma, papillary carcinoma of thyroid gland, colon, carcinoma, renal carcinoma, pancreatic carcinoma, ovarian carcinoma, head and neck squamous carcinoma.

Examples of the non-receptor tyrosine kinase include, but are not limited to, the Kit family (e.g., c-Kit), Src family, Fes family, JAK family, Fak family, Btk family, Syk/ZAP-70 family, and Abl family.

In particular, the Kit receptor tyrosine kinase is a transmembrane receptor that is expressed in a variety of different tissues and mediates pleiotropic biological effects through its ligand stem cell factor (SCF). Sporadic mutations of Kit as well as autocrine/paracrine activation mechanisms of the SCF/Kit pathway have been implicated in a variety of malignancies, where its primary contribution to metastases is in enhancing tumor growth and reducing apoptosis. For example, Kit is frequently mutated and activated in gastrointestinal stromal tumors (GISTs) and there is ligand-mediated activation of Kit in some lung cancers. Kit is a convenient target in Kit-induced tumors and inhibition of this receptor with the small molecule drug Gleevec® (imatinib mesylate, ST1571) in GIST has shown dramatic efficacy. Thus, the method of the present invention can be used to detect the Kit gene or its product to guide the selection of the patients who may be more responsive to therapeutic invention targeting Kit, such as a therapy of imatinib mesylate. For example, the present invention can be applied to detect c-Kit protein by using a primary antibody specific to c-Kit (anti-c-Kit antibody) in formalin-fixed, paraffin-embedded GIST tissue samples as an aid in the diagnosis of GIST in the context of the patient's clinical history, tumor morphology, and other diagnostic tests evaluated by a qualified pathologist.

Examples of the non-receptor tyrosine kinases from the Src family include, but are not limited to, Src, c-Src, v-Src, Yes, c-Yes, v-Yes, Fyn, Lyn, Lck, Bik, Hck, Fgr, c-Fgr, v-Fgr, p56lck, Tkl, Csk, and Ctk.

Examples of the inhibitors of the non-receptor tyrosine kinase from the Src family include, but are not limited to, SU101 and CGP 57418B.

Examples of the diseases associated with activity of the non-receptor tyrosine kinase from the Src family include, but are not limited to, breast cancer, carcinoma, myeloma, leukemia, and neuroblastoma.

Examples of the non-receptor tyrosine kinases from the Fes family include, but are not limited to, c-fes/fps, v-fps/fes, p94-c-fes-related protein, and Fer.

Examples of the diseases associated with activity of the non-receptor tyrosine kinase from the Fes family include, but are not limited to, tumor of mesenchymal origin and tumor of hematopoietic origin.

Examples of the non-receptor tyrosine kinases from the JAK family include, but are not limited to, Jak1, Jak2, Tyk2, and Jak3.

Examples of the inhibitors of the non-receptor tyrosine kinase from the JAK family include, but are not limited to, tyrphostin, member of CIS/SOCS/Jab family, synthetic component AG490, dimethoxyquinazoline compound, 4-(phenyl)-amino-6,7-dimethoxyquinazoline, 4-(4′-hydroxyphenyl)-amino-6,7-dimethoxyquinazoline, 4-(3′-bromo-4′-hydroxylphenyl)-amino-6,7-dimethoxyquinazoline, and 4-(3′,5′-dibromo-4′-hydroxylphenyl)-amino-6,7-dimethoxyquinazoline.

Examples of the diseases associated with activity of the non-receptor tyrosine kinase from JAK family include, but are not limited to, tumor of mesenchymal origin and tumor of hematopoietic origin.

Examples of the non-receptor tyrosine kinases from the Fak family include, but are not limited to, Fak and CAKB/Pyk2/RAFTK.

Examples of the inhibitors of the non-receptor tyrosine kinases from the Fak family include, but are not limited to, a dominant negative mutant S1034-FRNK; a metabolite FTY720 from Isaria sinclarii, and FAK antisense oligonucleotide ISIS 15421.

Examples of the diseases associated with abnormal activity of the non-receptor tyrosine kinases from Fak family include, but are not limited to, human carcinoma, metastasis-prone tumor, and tumor of hematopoietic origin.

Examples of the non-receptor tyrosine kinase from the Btk family include, but are not limited to, Btk/Atk, Itk/Emt/Tsk, Bmx/Etk, and Itk, Tec, Bmx, and Rlk.

Examples of the inhibitors of the non-receptor tyrosine kinases from Btk family include, but are not limited to, alpha-cyano-beta-hydroxy-beta-methyl-N-(2,5-dibromophenyl)propenamide.

Examples of the diseases associated with abnormal activity of the non-receptor tyrosine kinase from the Btk family include, but are not limited to, B-lineage leukemia and lymphoma.

Examples of the non-receptor tyrosine kinases from the Syk/ZAP-70 family include, but are not limited to, Syk and ZAP-70.

Examples of the inhibitors of the non-receptor tyrosine kinases from the Syk/ZAP-70 family include, but are not limited to, piceatannol, 3,4-dimethyl-10-(3-aminopropyl)-9-acridone oxalate, and acridone-related compound.

Examples of the diseases associated with abnormal activity of the non-receptor tyrosine kinases from the Syk/ZAP-70 family include, but are not limited to, benign breast cancer, breast cancer, and tumor of mesenchymal origin.

In yet another aspect, the present invention can be applied to detect the gene or gene product of a nuclear hormone receptor, such as estrogen, androgen, retinoid, vitamin D, glucoccoticoid and progestrone receptors. Nuclear hormone receptor proteins form a class of ligand activated proteins that, when bound to specific sequences of DNA serve as on-off switches for transcription within the cell nucleus. These switches control the development and differentiation of skin, bone and behavioral centers in the brain, as well as the continual regulation of reproductive tissues. Interactions between nuclear hormone receptors and their cognate ligands have been implicated in the initiation and development of various forms of cancer such as breast, prostate, bone, and ovarian cancer. Thus, by detecting the genes or gene products of the nuclear hormone receptors, diagnosis, prognosis and/or treatment of these diseases can be achieved.

Other than the specified diseases or conditions associated with the biomarkers described above, the present invention can also be used to detect the biomarkers in research, diagnosis, prognosis and/or treatment of diseases or disorders associated with undesirable, abnormal cell growth. Such diseases or disorders include, but are not limited to, restenosis (e.g. coronary, carotid, and cerebral lesions), benign tumors, a various types of cancers such as primary tumors and tumor metastasis, hematological disorders, abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Examples of benign tumors include hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

Specific types of cancers include, but are not limited to, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyoma tumor, cervical dysplasia and in situ carcinoma, neuroblastoma, retinoblastoma, soft tissue sarcoma, malignant carcinoid, topical skin lesion, mycosis fungoides, rhabdomyosarcoma, Kaposi's sarcoma, osteogenic and other sarcoma, malignant hypercalcemia, renal cell tumor, polycythermia vera, adenocarcinoma, glioblastoma multiforma, medulloblastoma, leukemias, Iymphomas, malignant melanomas, epidermoid carcinomas, and other carcinomas and sarcomas.

Examples of diseases associated with abnormal angiogenesis include, but are not limited to, rheumatoid arthritis, ischemic-reperfusion related brain edema and injury, cortical ischemia, ovarian hyperplasia and hypervascularity, (polycystic ovary syndrom), endometriosis, psoriasis, diabetic retinopaphy, and other ocular angiogenic diseases such as retinopathy of prematurity (retrolental fibroplastic), macular degeneration, corneal graft rejection, neuroscular glaucoma and Oster Webber syndrome.

Examples of retinal/choroidal neuvascularization include, but are not limited to, Bests diseases, myopia, optic pits, Stargarts diseases, Pagets disease, vein occlusion, artery occlusion, sickle cell anemia, sarcoid, syphilis, pseudoxanthoma elasticum carotid abostructive diseases, chronic uveitis/vitritis, mycobacterial infections, Lyme's disese, systemic lupus erythematosis, retinopathy of prematurity, Eales disease, diabetic retinopathy, macular degeneration, Bechets diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the angle) and diseases caused by the abnormal proliferation of fibrovascular or fibrous tissue including all forms of proliferative vitreoretinopathy.

Examples of comeal neuvascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, diabetic retinopathy, retinopathy of prematurity, corneal graft rejection, Mooren ulcer, Terrien's marginal degeneration, marginal keratolysis, polyarteritis, Wegener sarcoidosis, Scieritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections and Kaposi sarcoma.

The present invention can also be applied to detect variation in nucleic acid sequences. The ability to detect variations in nucleic acid sequences is of great importance in the field of medical genetics: the detection of genetic variation is essential, inter alia, for identifying polymorphisms for genetic studies, to determine the molecular basis of inherited diseases, to provide carrier and prenatal diagnosis for genetic counseling and to facilitate individualized medicine. Detection and analysis of genetic variation at the DNA level has been performed by karyotyping, analysis of restriction fragment length polymorphisms (RFLPs) or variable nucleotide type polymorphisms (VNTRs), and more recently, analysis of single nucleotide polymorphisms (SNPs). See e.g. Lai E, et al., Genomics, 1998, 15;54(1):31-8; Gu Z, et al., Hum Mutat. 1998;12(4):221-5; Taillon-Miller P, et al., Genome Res. 1998;8(7):748-54; Weiss K M., Genome Res. 1998;8(7):691-7; Zhao LP,.et al., Am J Hum Genet. 1998; 63(1):225-40.

According to the present invention, for example, a nucleic acid probe for a SNP can be labeled with an enzyme, e.g., peroxidase, covalently or non-covalently, and hybridized to the site of the SNP. The enzyme is then utilized as previously described to deposit metal at the site of the SNP specifically. Multiple probes can be utilized for detecting multiple SNPs in a population of target polynucleotides in parallel as well as by following the general principles described herein.

In other embodiments, the present invention can be used to detect chromosomal gene position and/or abnormalities. In some embodiments, a sample containing an unknown chromosome complement is labeled with deposited metal and at least a second detectable signal (e.g., FITC) using probes that specifically hybridize to the genetic sequences of interest. The relative positions of the first and second labels in the chromosome complement are then ascertained. These relative locations have a predetermined normal geometric pattern in the chromosome complement. In other embodiments, genetic sequences in close proximity to each other on the same chromosome may be detected by determining the relative positions of the first and second labels, even in phases of the cell cycle where condensed chromosomes are not present.

As used herein, the term “predetermined pattern” refers to a previously identified geometrical relationship between probe target position and number on a normal chromosome complement and modified patterns on abnormal chromosome complements. When appropriate, a “predetermined pattern” includes not only the spatial relationship and quantity of different probe targets on interphase nuclei but also on metaphase chromosome complements that are stained uniformly or banded by any chromosome banding procedure. Generally, single adjacent probe targets on normal chromosomes carrying each normal gene type of interest will reflect the “predetermined normal pattern” in some embodiments of the present invention.

### 3. Multicolor Detection of Multiple Biomarkers

Site-specific enzymatic deposition of metal described above can be used in conjunction with other detection methods to detect multiple targets in a sample.

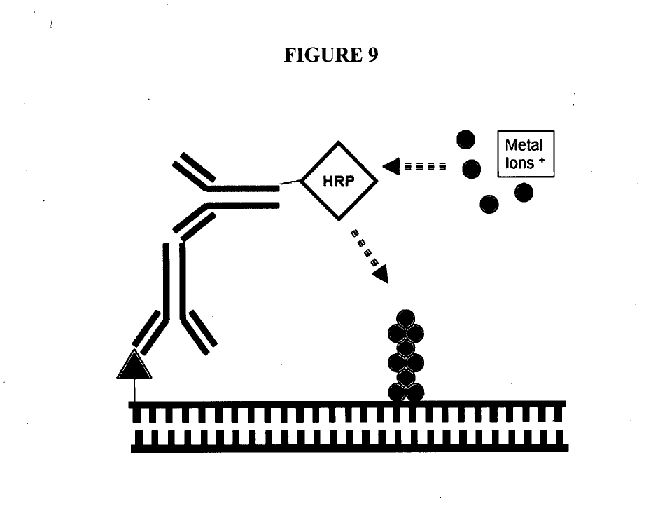

FIG. 9 is a graphic depiction of the deposition of metal in the vicinity of a target molecule. In this depiction a DNA probe labeled with a hapten is bound to a target sequence. The hapten is bound by an anti-hapten primary antibody. The primary antibody is bound by a horseradish peroxidase-conjugated secondary antibody. Metal ions in solution are reduced to elemental metal in the vicinity of the bound enzyme.

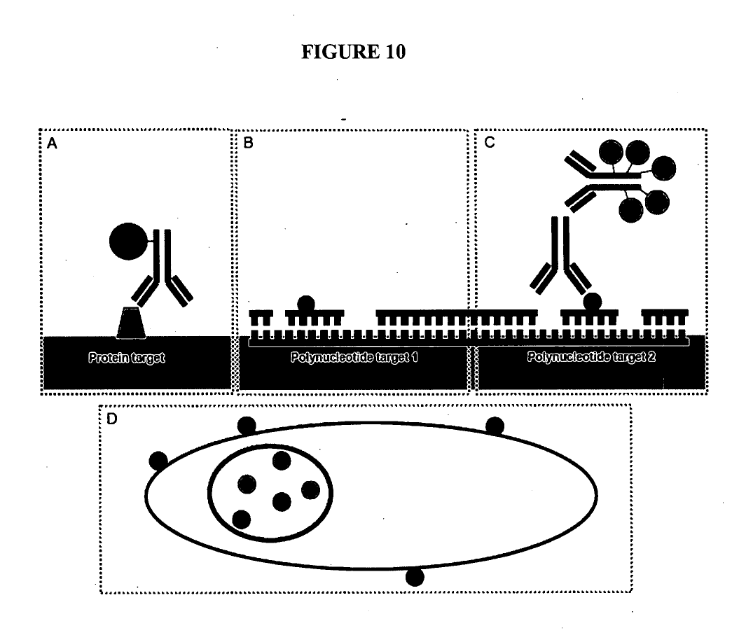

FIG. 10 is a graphic depiction of the metal deposition used in combination with other signals. Panel A shows a surface protein bound by an alkaline-phosphatase-conjugated antibody which produces a calorimetric signal upon addition of an appropriate substrate. Panel B shows a DNA probe labeled with a fluorescent marker hybridized to a target sequence. Panel C shows a DNA probe labeled with a hapten bound to a target sequence. The hapten is bound by an anti-hapten primary antibody. The primary antibody is bound by a secondary antibody multiply conjugated with horseradish peroxidase enzymes, which leads to the deposition of metal in the vicinity of the bound antibody. D depicts a cell to which the three different probes are bound. Because of the localization of each signal, concurrent detection of all three target molecules is possible.

For example, the inventive method can be used to determine the presence and/or amount of a target biomarker while alternate methods are used to determine the presence and/or amount of other target biomarkers. Examples of such alternate methods are described in detail below.

Many conventional detection methods utilize enzymes. The types of enzyme substrates popularly used for sensitive detection are typically colorimetric, radioactive, fluorescent or chemiluminescent. Conventional colorimetric substrates produce a new color (or change in spectral absorption) upon enzyme action on a chromogenic substrate. This type of detection is advantageous in that the chromogens produced are easily detected by light-based microscopy or with spectral equipment. The cost of equipment for detection is also generally less than with other methods; for example in pathology, the brown color produced by the enzyme horseradish peroxidase acting on the substrate 3,3′-diaminobenzidine (DAB), requires only a simple bright field light microscope for observation of biopsied sections. Other chromogens which can be used in conjunction with horseradish peroxidase include, but are not limited to, 3-Amino-9-ethylcarbazole (AEC) and Bajoran Purple. Other chromogens which can be used in conjunction with alkaline phosphatase include, but are not limited to, Fast Red and Ferangi Blue. Numerous chromogens are available to a person having ordinary skill in the art, and are commercially available through catalogs provided by companies such as Thermo Fisher Scientific.

Conventional radioactive substrates can enzymatically release or fix radioactivity for measurement. Although sensitive, this type of detection is becoming less popular due to the risks of handling and disposing of radioactive material, and other methods now rival or exceed its sensitivity. Radioactive labeling for histochemical uses and autoradiography, typically require months to expose films, due to low specific activity, which is another disadvantage.

Fluorescent substrates are popular because they are reasonably sensitive, generally have low backgrounds, and several differently colored fluorophores can be used simultaneously.

Chemiluminescence is based upon use of substrates that have sufficiently high chemical bond energies so that when the bonds are broken by an enzyme, energy is released in the form of visible light. This method has gained popularity due to the low background and very high sensitivity obtainable using photomultipliers, avalanche diodes or other sensitive light detectors. Alternatively photographic film can be used as a detection means.

One of skill in the art will understand that detectable moieties used in combination with the site-specific deposition of metal can be any material having a detectable physical or chemical property. Such detectable labels have been well developed in the field of gels, columns, and solid substrates, and in general, labels useful in such methods can be applied to the present invention. Thus, a label is any composition detectable by spectroscopic, photochemical, biochemical, immunochemical, electrical, optical or chemical means. Furthermore, it will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule. Bruchez et al. (1998) Science 281: 2013-2016. Similarly, highly fluorescent quantum dots (zinc sulfide-capped cadmium selenide) have been covalently coupled to biomolecules for use in ultrasensitive biological detection. Warren and Nie (1998) Science 281: 2016-2018.

Useful labels in the present invention include fluorescent dyes (e.g., fluorescein isothiocyanate, Texas red, rhodamine, and the like), enzymes (e.g., LacZ, CAT, horseradish peroxidase, alkaline phosphatase, I²-galactosidase, β-galactosidase, and glucose oxidase, acetylcholinesterase and others, commonly used as detectable enzymes), quantum dot-labels, chromophore-labels, enzyme-labels, affinity ligand-labels, electromagnetic spin labels, heavy atom labels, probes labeled with nanoparticle light scattering labels or other nanoparticles, fluorescein isothiocyanate (FITC), TRITC, rhodamine, tetramethylrhodamine, R-phycoerythrin, Cy-3, Cy-5, Cy-7, Texas Red, Phar-Red, allophycocyanin (APC), epitope tags such as the FLAG or HA epitope, and enzyme tags such as and hapten conjugates such as digoxigenin or dinitrophenyl, or members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue, dichlorotriazinylamine fluorescein, dansyl chloride, phycoerythrin, fluorescent lanthanide complexes such as those including Europium and Terbium, molecular beacons and fluorescent derivatives thereof, a luminescent material such as luminol; light scattering or plasmon resonant materials such as gold or silver particles or quantum dots; or radiolabels including ¹⁴C, ¹²³I, ¹²⁴I, ¹³¹I, ¹²⁵I, Tc99m, ³²P, ³⁵S or ³H; or spherical shells, and probes labeled with any other signal generating label known to those of skill in the art, as described, for example, in Principles of Fluorescence Spectroscopy, Joseph R. Lakowicz (Editor), Plenum Pub Corp, 2nd edition (July 1999) and the 6^{th} Edition of the Molecular Probes Handbook by Richard P. Hoagland.

Semidconductor nanocrystals such as quantum dots (i.e., Qdots) described in U.S. Pat. No. 6,207,392, are commercially available from Quantum Dot Corporation and include nanocrystals of Group II-VI semiconductors such as MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, and HgTe as well as mixed compositions thereof; as well as nanocrystals of Group Ill-V semiconductors such as GaAs, InGaAs, InP, and InAs and mixed compositions thereof. The use of Group IV such as germanium or silicon, or the use of organic semiconductors, may also be feasible under certain conditions. The semiconductor nanocrystals may also include alloys comprising two or more semiconductors selected from the group consisting of the above Group III-V compounds, Group II-VI compounds, Group IV elements, and combinations of same. Examples of labels can also be found in U.S. Pat. Nos. 4,695,554; 4,863,875; 4,373,932; and 4,366,241. Colloidal metals and dye particles are disclosed in U.S. Pat. Nos. 4,313,734 and 4,373,932. The preparation and use of non-metallic colloidals are disclosed in U.S. Pat. No. 4,954,452. Organic polymer latex particles for use as labels are disclosed in U.S. Pat. No. 4,252,459.

Embodiments for which a target biomarker is a nucleic acid will typically involve production of a probe specific to the target. Probes may be generated and chosen by several means including mapping by in situ hybridization [Landegent et al, Nature 317:175-177, 1985], somatic cell hybrid panels [Ruddle & Creagan, Ann. Rev. Genet. 9:431, 1981], or spot blots of sorted chromosomes [Lebo et al, Science 225:57-59, 1984]; chromosomal linkage analysis [Ott, Analysis of Human Genetic Linkage, Johns Hopkins Univ Press, pp. 1-197, 1985]; or cloned and isolated from sorted chromosome libraries from human cell lines or somatic cell hybrids with human chromosomes [Deaven et al, Cold Spring Harbor Symp. LI:159-168, 1986; Lebo et al. Cold Spring Harbor Symp. LI: 169-176], radiation somatic cell hybrids [Cox et al, Am. J. Hum. Genet. 43:A141, 1988], microdissection of a Chromosome region [Claussen et al, Cytometry 11:suppl 4 p. 12, 1990], (all of which are incorporated by reference), or from yeast artificial chromosomes (YACs) identified by PCR primers specific for a unique chromosome locus (sequence tagged site or STS) or other suitable means like an adjacent YAC clone.

Probes may be either RNA or DNA oligonucleotides or polynucleotides and may contain not only naturally occurring nucleotides but their analogs like digoxygenin dCTP, biotin dcTP 7-azaguanosine, azidothymidine, inosine, or uridine. Probes may be genomic DNA, cDNA, or viral DNA cloned in a plasmid, phage, cosmid, YAC, or any other suitable vector. Probes may be cloned or synthesized chemically. When cloned, the isolated probe nucleic acid fragments are typically inserted into a replication vector, such as lambda phage, pBR322, M13, pJB8, c2RB, pcoslEMBL, or vectors containing the SP6 or T7 promoter and cloned as a library in a bacterial host. General probe cloning procedures are described in Arrand J. E., Nucleic Acid Hybridization A Practical Approach, Hames B. D., Higgins, S. J., Eds., IRL Press 1985, pp. 17-45 and Sambrook, J., Fritsch, E. F., Maniatis, T., Molecular Cloning A Laboratory Manual, Cold Spring Harbor Press, 1989, pp. 2.1-3.58, both of which are incorporated herein by reference.

Alternatively, oligonucleotide probes may be synthesized chemically with or without fluorochromes, chemically active groups on nucleotides, or labeling enzymes. Other methods may also be used to synthesize oligonucleotide probes, e.g., the solid phase phosphoramidite method that produces probes of about 15-250 bases. Methods are detailed in Caruthers et al., Cold Spring Harbor Symp. Quant. Biol., 47:411-418, 1982, and Adams, et al., J. Am. Chem. Soc., 105:661, 1983, both of which are incorporated herein by reference. Polymerase chain reaction can also be used to obtain large quantities of isolated probes. The method is outlined in U.S. Pat. No. 4,683,202, incorporated herein by reference.

It is well understood in the art that when synthesizing a probe for a specific nucleic acid target, the choice of nucleic acid sequence will determine target specificity. Typically, probes have sufficient complementarity to their target polynucleotides so that stable and specific binding occurs between the chromosome and the probe. The degree of homology required for stable hybridization varies with the stringency of the hybridization medium and/or wash medium. Preferably completely homologous probes are employed in the present invention, but persons of skill in the art will readily appreciate that probes exhibiting lesser but sufficient homology can be used in the present invention.

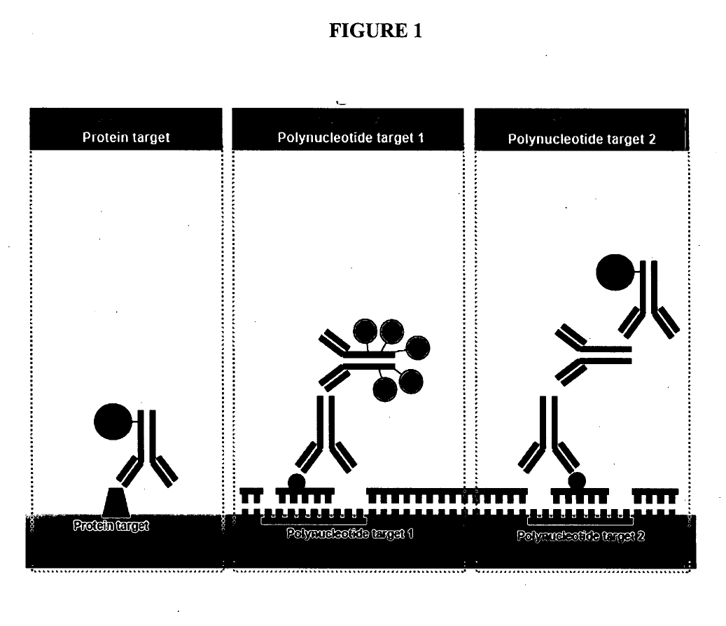

A general schematic representation of multicolor detection of target biomarkers using antibodies is shown in FIGS. 1 and 10. FIG. 1 shows detection of a protein target using a labeled primary antibody. The label on this primary antibody can be any label known in the art and/or as described herein (e.g. a fluorescent indicator, an enzyme which produces a calorimetric signal upon addition of appropriate substrate, or a radioactive isotope). FIG. 1 further shows detection of a first polynucleotide target utilizing both a primary antibody and a secondary antibody. In this embodiment, the secondary antibody is conjugated to multiple enzymes (e.g., horseradish peroxidase) or multiple labels. Generally, such multiple conjugation will increase the signal from such a bound antibody. FIG. 1 also demonstrates detection of a second nucleotide target using a primary antibody and two secondary antibodies. One of skill in the art will recognize that the primary antibody may be specific to a particular target molecule or may be specific to a hapten or other target bound to a target-specific probe. One of skill in the art will also recognize that this method could be used to detect multiple protein targets and/or multiple polynucleotide targets.

FIG. 10 shows direct and indirect labeling of target biomarkers using unlabeled and/or labeled antibodies and using direct probes. FIG. 10 shows detection of a protein target using a labeled primary antibody, detection of a first polynucleotide target using a labeled nucleotide probe and detection of a second polynucleotide target using a hapten-conjugated nucleotide probe, a primary antibody specific to the hapten, and a multiple-conjugate secondary antibody specific to the primary antibody. By utilizing three different labels/indicators it is possible to distinguish between the signals indicating the presence/level of the different target molecules. An example of a cell in which three distinguishable signals are present is shown graphically in panel D.

An antibody can encompass monoclonal antibodies, polyclonal antibodies, antibody fragments (e.g., Fab, Fab′, F(ab′)₂,.Fv, Fc, etc.), chimeric antibodies, single chain (ScFv), mutants thereof, fusion proteins comprising an antibody portion, and any other polypeptide that comprises an antigen recognition site of the required specificity. The antibodies may be murine, rat, rabbit, chicken, human, or of any other origin (including humanized antibodies).

The labeled antibodies can be conjugated to any detection means described above. In most embodiments, at least one of the antibodies is conjugated to an enzyme which will allow for the enzymatic deposition of metal. The other labeled antibody or antibodies will be conjugated to a substance which allows for detection of signals, including, but not limited to colorimetric, radioactive, fluorescent and/or chemiluminescent signals. For example, in one embodiment, at least one antibody used to detect one target biomarker will be conjugated with an enzyme, such as alkaline phosphatase, which will allow for the colorimetric detection of specific binding upon addition of appropriate substrates for alkaline phosphatase. In some embodiments, multiple enzymes (e.g., horseradish peroxidase) are conjugated to at least one of the antibodies used to detect a target biomarker (e.g., primary, secondary, tertiary, etc.). Methods of constructing multiple-conjugate antibodies are known in the art and a preferred method is described in U.S. application Ser. No. 11/413,418 (filed Apr. 27, 2006), the relevant parts of which are herein incorporated by reference.

In embodiments involving detection of a nucleic acid biomarker, a directly detectable substance can be incorporated into the nucleic acid probe. Also, as described above, a nucleic acid probe may be covalently or non-covalently labeled with an enzyme (e.g., peroxidase) and utilized as previously described to deposit metal at the site of binding.

Typically, a different signal will be used to label target biomarkers, thus allowing for detection of multiple biomarkers in the same sample. For example, the following three detection mechanisms can be used in some embodiments: 1) the first biomarker will be directly or indirectly detected with a colorimetric indicator; 2) the second biomarker will be directly or indirectly detected with a metal deposition indicator, and; 3) the third biomarker is directly or indirectly detected with a fluorescent indicator. One of skill in the art will understand that, when used in combination to detect multiple biomarkers, the individual labels will typically be readily distinguishable from each other. In some embodiments, two, three, four, five, six, seven, eight, nine, ten or more individual biomarkers will be detected.

In one embodiment, the detection of individual target biomarkers can be performed sequentially. In other embodiments, detection of individual target biomarkers can be performed concurrently or substantially concurrently. In still other embodiments, a combination of sequential and concurrent or substantially concurrent detection of individual target biomarkers can be performed. In embodiments involving detection of biomarkers in cells, cell fragments, tissues, and/or biological or environmental samples, appropriate stains (e.g. hematoxylin, crystal violet, Coomassie blue, Nuclear Fast Red, Methyl Green, Methyl Blue, etc.) can be used to aid examination.

Detection of biomarkers using different signals has been described using fluorescent markers (FISH) (U.S. Pat. No. 5,665,540). Catalytic silver deposition following binding of Nanogold probes has been used to detect target molecules (Hainfeld, J. F. and F. R. Furuya, (1995) “Immunogold-Silver Staining: Principles, Methods and Applications,” in Silver Enhancement of Nanogold and Undecagold, M. A. Hayat (Ed.); pp. 71-96), as has catalytic gold deposition following binding of Nanogold probes (Hainfeld, J. F. and R. D. Powell, (2002) “Silver- And Gold-Based Autometallography Of Nanogold” in “Gold and Silver Staining: Techniques in Molecular Morphology”, Hacker and Gu (Eds.), pp. 29-46.

Each of these approaches has specific drawbacks. Specifically, FISH requires fluorescent optics and high magnification. Furthermore, cellular morphology is often difficult to determine. Fluorescently labeled specimens can fade and cannot be permanently archived. Silver-enhanced Nanogold probe labeling is time consuming, has restrictive reaction conditions, is light sensitive and shows some non-specificity. Gold-enhanced Nanogold probe labeling does not allow for quantitative interpretation. The present invention overcomes all of these limitations and has the unexpected result of being easily adapted to automated methods, improving workflow, efficiency, and throughput. Additionally, the present invention yields high quality, quantitative results that are easily interpreted.

Having generally described certain aspects of the invention, the following examples are included for purposes of illustration so that the invention may be more readily understood and are in no way intended to limit the scope of the invention unless otherwise specifically indicated.

### EXAMPLES

### Example 1

### Detection of Single Copy of HER2 Gene in Tissue Using SISH Detection

In this example, the method of the present invention was used to sensitively and selectively detect a single copy of a gene in situ, such as HER2 gene in normal as well as in breast cancer tissue, using Silver in situ hybridization (“SISH”).

Formalin-fixed, paraffin-embedded (FFPE) human breast carcinoma containing normal breast epithelium was used as a positive control. The slides containing the FFPE tissue were prepared and stained automatically by using an automated staining system BenchMark™ XT (Ventana Medical Systems, Inc., Tucson, Ariz.) operated in accordance with standard procedures.

The pre-programmed protocol “XT SISH iVIEW™ SILVER” was used to prepare and stain all tissues via automated in situ hybridization. The deparaffinization option was selected for 20 minutes at 75° C. under the EZ Prep™ solution (Ventana P/N 950-102). The solution is applied through the rinse nozzles which leaves approximately 200-300 ul of residual volume of the solution on the slide. The slide is then incubated at temperature for 4 minutes. This process cycles 5 times. The cell conditioning option was also selected, and was performed under CC2 reagent (Ventana P/N 950-123, which is a high pH retrieval solution that serves to solubilize the cell membrane which allows genetic material to be accessible for target probe hybridization). The solution is applied into a residual volume of EZ prep and heated at 90° C. for 20 minutes. Following cell conditioning, protease digestion was selected using ISH Protease 3 (Ventana P/N 780-4149, a type VIII protease isolated from Bacillus licheniformus). Approximately 100 ul of ISH Protease 3 is dispensed into 200-300 ul of reaction buffer and incubated for 4 minutes at 37° C. The detection system applied to the tissue was the iVIEW™ SILVER (Ventana, P/N 790-098) detection system described herein. Briefly, a biotinylated HER2 gene probe (Ventana, P/N 780-2840) spanning the full coding sequence of the HER2 gene was hybridized using stringent, hybridization conditions to the HER2 gene in both normal and carcinoma tissues. Successively, an anti-biotin rabbit polyclonal antibody, then a goat anti-rabbit-Horseradish Peroxidase conjugate antibody is incubated with the tissue. Prior to application of chromogenic reagents, slides were washed with an un-buffered solution containing a surfactant and water. Appoximately 100 μl of the wash remains on the slide prior to reagent application, 100 μl each of Reagents A (0.18% silver acetate), B (0.18% hydroquinone), and C (0.07% hydrogen peroxide), were dispensed onto the slide. Reagent A is applied first and incubated for 4 minutes at 37° C. Without rinsing, Reagent B is applied to the slide and allowed to incubate an additional 4 minutes at 37° C. Finally, Reagent C is added to the pool of Reagent A and B and incubated for a final 4 minutes at 37° C.

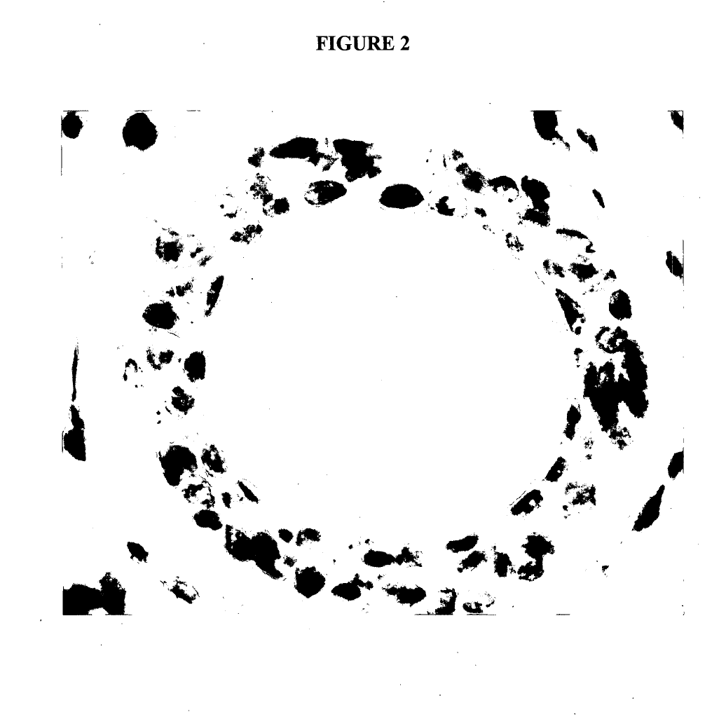

FIG. 2 is a photomicrograph at 100× showing dots of deposited silver metal as discrete single copies of HER2 gene in a 4 micron-section of normal breast duct tissue. There are one to two signals per cell, the normal complement. The tissue is counterstained violet with Hematoxylin II, a lipophilic biological stain. Hematoxylin II stains cell nuclei violet through the binding of a mordant dye complex to nucleic acids and histone proteins of the heterochromatin (Ventana P/N 790-2208). Bluing Reagent, a high pH metal salt and carbonate solution was applied to the tissue sample after rinsing and reacts with the Hematoxylin to produce a blue counterstain (Ventana P/N 760-2037). Thus, FIG. 2 shows that by using the method of the present invention, a single copy of HER2 gene can be detected in normal breast tissue.

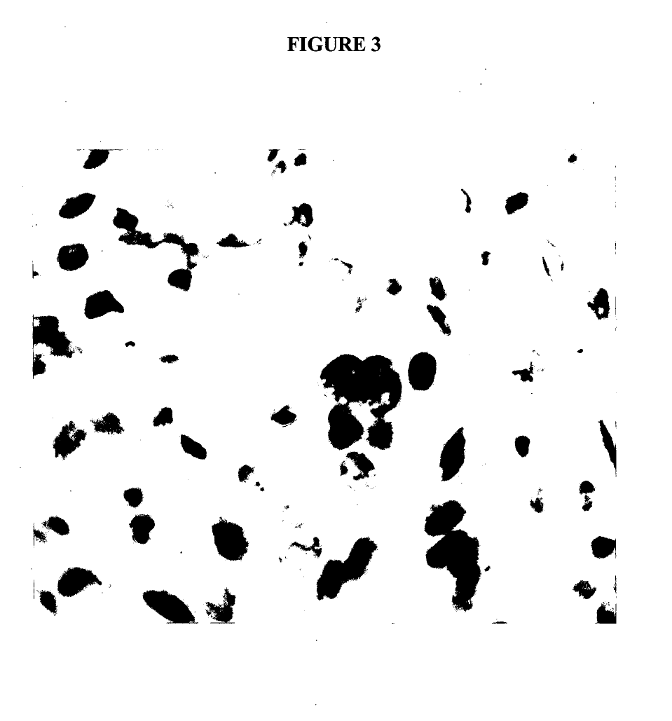

FIG. 3 is a photomicrograph at 100× showing dots of deposited silver metal as discrete single copies of HER2 gene in a 4 micron-section of breast tumor tissue that is not amplified for HER2. As shown in FIG. 3, the normal complement of 2 single copies of the HER2 gene is readily distinguishable. Thus, FIG. 3 shows that by using the method of the present invention, a single copy of HER2 gene can be detected in breast tumor tissue with non-amplified HER2 gene.

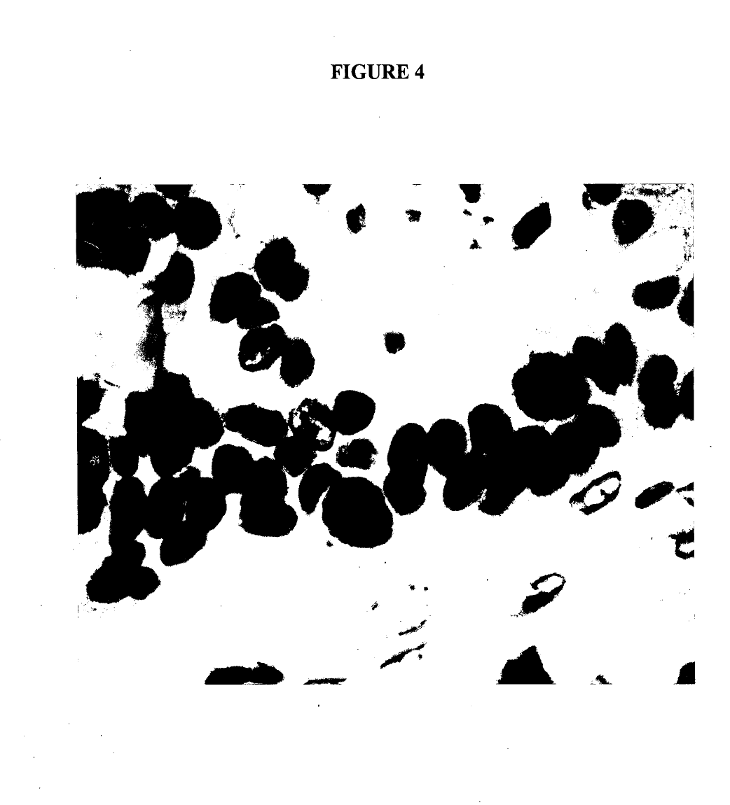

FIG. 4 is a photomicrograph at 100× showing dots of deposited silver metal as numerous copies of HER2 gene in a 4 micron-section of breast tumor tissue with low levels of HER2 gene amplification, from 3 to 5 copies of HER2 gene present in the tumor cells. Thus, FIG. 4 shows that by using the method of the present invention, multiple copies of HER2 gene can be detected in breast tumor tissue with low levels of HER2 gene amplification.

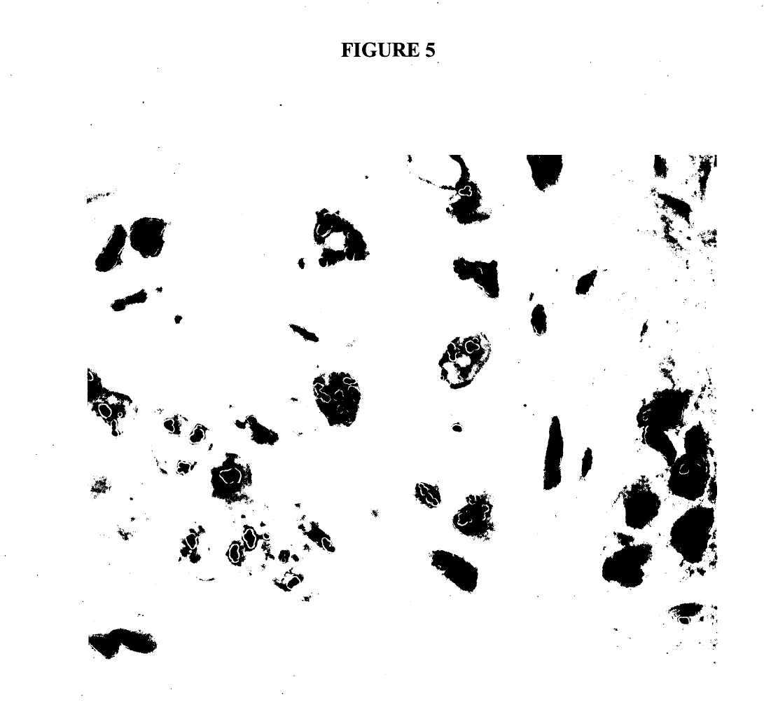

FIG. 5 is a photomicrograph at 100× showing dots of deposited silver metal as many copies of HER2 gene in a 4 micron-section of breast tumor tissue with relatively higher levels of HER2 gene amplification (than that of breast tumor tissue shown in FIG. 4), with 6 or greater copies of HER2 gene present in the tumor cells. Due to the high levels of HER2 gene amplification, the silver metal signals begin to become unresolved and appear to fuse into large clusters of silver metal. Thus, FIG. 5 shows that by using the method of the present invention, many copies of HER2 gene can be detected in breast tumor tissue with high levels of HER2 gene amplification and the size of the silver metal dots corresponds to the level of amplification of the targeted gene.

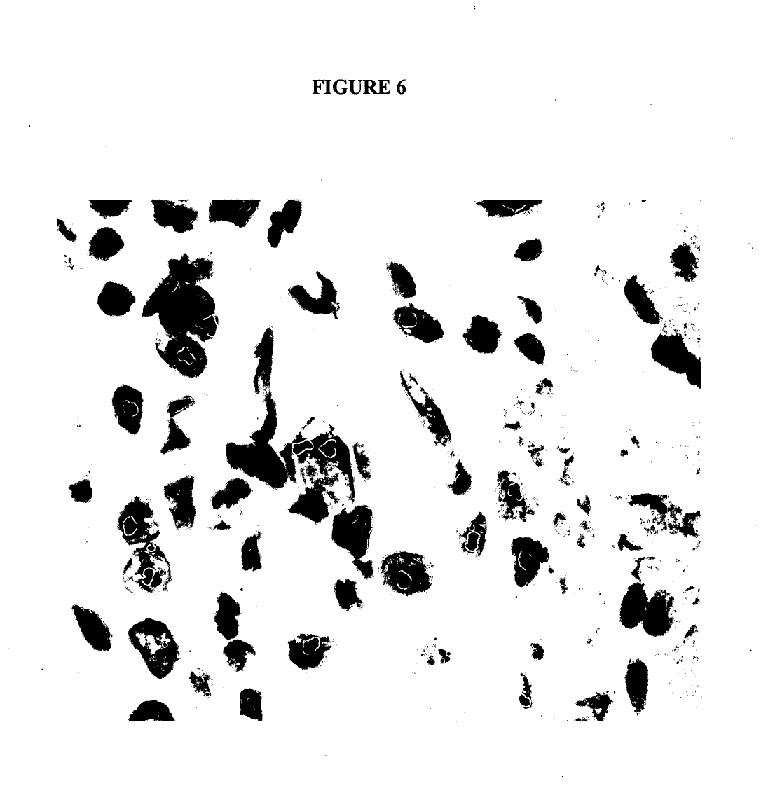

FIG. 6 is a photomicrograph at 100× showing dots of deposited silver metal as many copies of HER2 gene in a 4 micron-section of breast tumor tissue with even higher levels of HER2 gene amplification (than that of breast tumor tissue shown in FIG. 5). Due to the extremely high level of HER2 gene amplification, the silver metal signal is so intense it becomes fused, forming a big dot of silver metal. This experiment further demonstrated that the method of the present invention can sensitively and selectively detect not only single copies of a targeted gene but also multiple copies of the targeted gene in situ; and the intensity of the metal signal corresponds to the level of amplification of the targeted gene.

In another experiment, the concentrations of the Reagent A, B and C and the silver staining reaction conditions were adjusted to further improve the sensitivity of the assay for detecting the gene copy of the HER2 gene and to further reduce the background staining. Briefly, a DNP (2,4-dinitrophenyl) labeled HER2 gene probe (Ventana Medical Systems Product No. 780-4332) was formulated in 80% Hybrizol containing 2 mg/ml Human DNA for use in assays. Silver in situ hybridization (“SISH”) according to the present invention was performed on formalin-fixed, paraffin-embedded 4 micron-thick human breast tumor tissue sections mounted on glass microscope slides using the automated ISH protocol, available in conjunction with the BenchMark™ series instrument (Ventana Medical Systems, Tucson, Ariz.). In brief, after paraffin removal and protease treatments, hybridization with the DNP-labeled HER2 specific probe was carried out for 2 hours at 52° C. in 2×SSC and 23% formamide. After washing with 2×SSC a rabbit anti-DNP antibody (2 μg/ml) was applied, followed by a 20 minute incubation at 37° C. After washing, a goat HRP-conjugated anti-rabbit antibody was applied (15 μg/ml) and incubated for an additional 20 minutes. After washing with 100 mM citrate buffer pH 3.9, a solution of silver acetate (3.68 mg/ml) was applied and incubated for 4 minutes. It was washed again, and a second incubation with silver acetate (3.68 mg/ml)) was applied to the slide and incubated for 4 minutes. Without washing, a hydroquinone solution (1.78 mg/ml in 0.1 M citrate pH 3.8) of equal volume to that of the silver acetate solution was applied to the slide, followed by a solution of 0.09% w/v hydrogen peroxide of equal volume to that of the silver acetate solution, resulting in the final concentration of silver acetate being 1.23 mg/ml (or 0.123% w/v), hydroquinone 0.6 mg/ml (or 0.06% w/v), and 0.03% w/v hydrogen peroxide. After 12 minutes the slides were washed and dried for mounting. After silver staining, a nuclear counter stain (Hematoxylin, Ventana PN 790-2208) was applied, with bluing reagent (Ventana Medical Systems Product No. 760-2037) according to manufacturer's instructions.

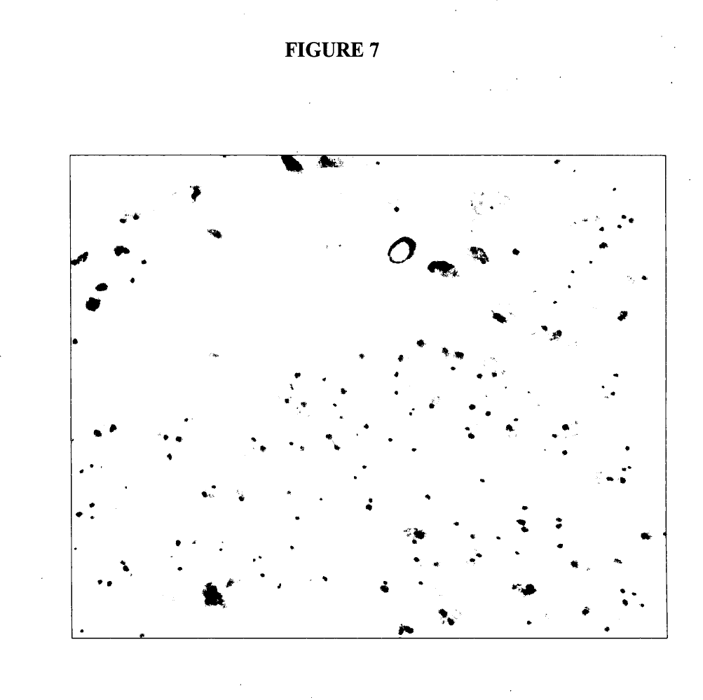

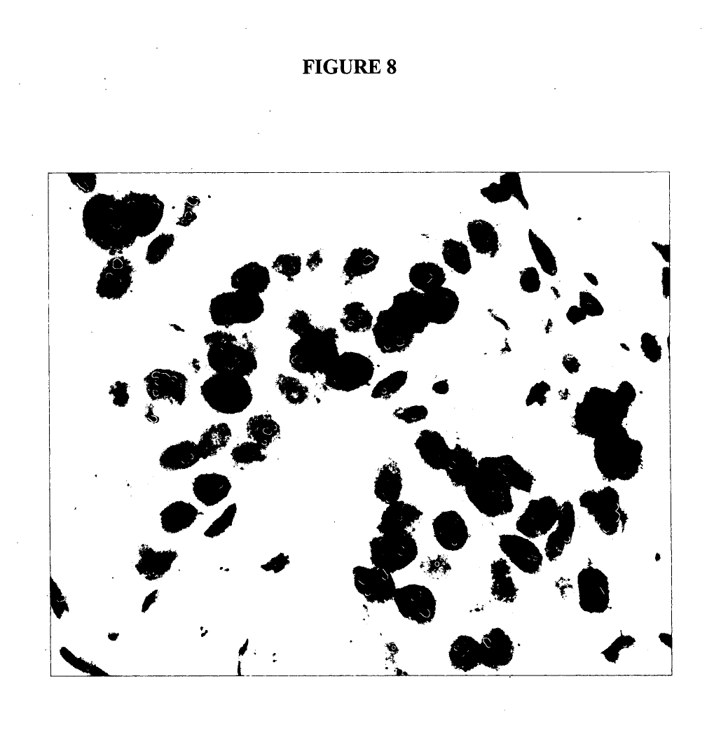

Exemplary SISH results are illustrated in FIGS. 7 and 8, which are lightfield photomicrographs. FIG. 7 shows a sample in which the HER2 target sequence is unamplified (diploid). Cells in this sample exhibit 2 or fewer hybridization signals (which appear as dark dots). FIG. 8 shows a sample in which the HER2 target sequence is amplified to many times the diploid copy number. Hybridization signals appear as multifocal aggregates of black dots. Compared to FIGS. 2 and 3, discrete single copies of HER2 gene shown in FIG. 7 are even more distinguishable with lower background levels.

### Example 2

### Detection of Single Copy of HER2 Gene in Tissue with SISH

In yet another experiment, the method of the present invention was used to sensitively and selectively detect a single copy of a gene in situ, such as normal HER2 genes as well as amplified HER2 genes, using a repeat-depleted HER2 probe with SISH detection.

Formalin-fixed, paraffin-embedded (FFPE) human breast carcinoma cell line xenograft tumors were used for assay optimization. The slides containing the FFPE tumor sections were prepared and stained using an automated staining system BenchMark™ XT (Ventana Medical Systems, Inc., Tucson, Ariz.) operated in accordance with standard procedures.

The pre-programmed protocol “XT SISH iVIEW™ SILVER” was used to prepare and stain all tissues via automated in situ hybridization. The deparaffinization option was selected for 20 minutes at 75° C. under the EZ Prep™ solution (Ventana P/N 950-102). The solution was applied through the rinse nozzles which leaves approximately 200-300 μl of residual volume of the solution on the slide. The slide was then incubated at 75° C. for 4 minutes. The solution application and incubation was repeated 4 additional times. The cell conditioning option was also selected, and was performed using CC2 reagent (Ventana P/N 950-123; which is a high pH retrieval solution that serves to solubilize the cell membrane which allows genetic material to be accessible for target probe hybridization). The solution was applied into a residual volume of EZ prep and heated at 90° C. for 20 minutes. Following cell conditioning, protease digestion was selected using ISH Protease 3 (Ventana P/N 780-4149 a type VIII protease isolated from Bacillus licheniformus). Approximately 100 μl of ISH Protease 3 was dispensed into 200-300 ul of Reaction Buffer (Ventana P/N 950-300) and incubated for 4 minutes at 37° C.

Detection of the HER2 genes by.silver deposition was performed essentially as described in the previous example, except the HER2 probe was constructed by dinitrophenol (DNP) labeling a composite repeat depleted (CORD) probe. CORD probes are described in detail in U.S. Provisional Pat. App. Ser. No. 60/841,896, filed Sep. 1, 2006, incorporated in its entirety herein by reference. Briefly, CORD probes are completely or substantially completely depleted of repetitive nucleic acid elements (e.g. Alu repeats, LI repeats, and Alpha satellite DNA), and correspond on a segment by segment basis to unique coding or non-coding elements of the target sequence.

DNP-labeled HER2 gene probe (Ventana P/N 780-4332) spanning the full coding sequence of the HER2 gene was hybridized using stringent hybridization conditions to the HER2 gene in xenograft tumor sections. Next, the iVIEW™ SILVER (Ventana, P/N 790-098) detection system was applied to the tissue. Successively, an anti-DNP rabbit monoclonal antibody, then a goat anti-rabbit-Horseradish Peroxidase conjugate antibody is incubated with the tissue. Prior to application of chromogenic reagents, slides were washed with an unbuffered solution containing a surfactant and water (SISH Wash, Ventana P/N 780-002). Approximately 100 μl of the wash remained on the slide prior to reagent application, 100 μl each of Silver Chromogen A (0.36% silver acetate), Silver Chromogen B (0.18% hydroquinone), and Silver Chromogen C (0.09% hydrogen peroxide), were dispensed onto the slide. Silver Chromogen A was applied first and incubated for 4 minutes at 37° C. Without rinsing, Silver Chromogen B was applied to the slide and allowed to incubate an additional 4 minutes at 37° C. Finally, Silver Chromogen C was added to the pool of Silver Chromogen A and B and incubated for a final 12 minutes at 37° C. The tissue was counterstained blue with Hematoxylin II (Ventana N/P 790-2208) and Bluing Reagent (Ventana P/N 760-2037).

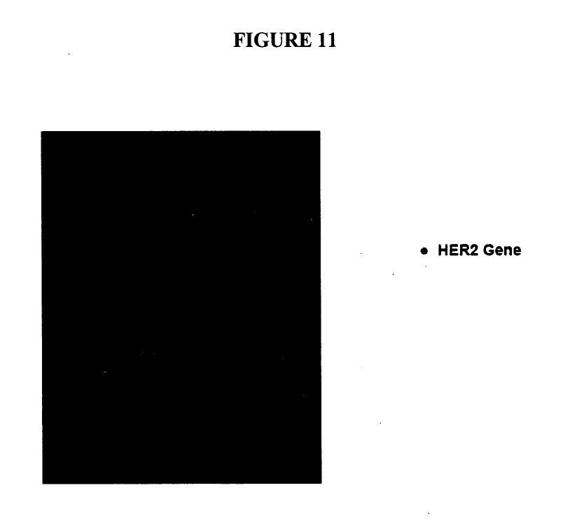

FIG. 11 is a photomicrograph at 100× showing dots of deposited silver metal as discrete single copies of HER2 gene in a 5 μm-section of formalin-fixed, paraffin-embedded MCF7 (A highly rearranged, near triploid human breast carcinoma cell line) xenograft tumor. There are one to three signals per cell, dependent on the cell cycle stage and how each cell was cut.

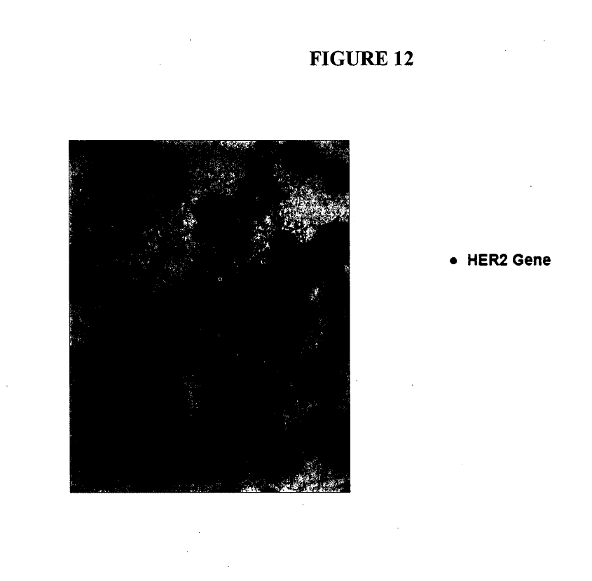

FIG. 12 is a photomicrograph at 100× showing dots of deposited silver metal as discrete single copies of HER2 gene in a 5 μm-section of formalin-fixed, paraffin-embedded ZR-75-1 (A highly rearranged, near triploid human breast carcinoma cell line) xenograft tumor. There are one to three signals per cell, dependent on the cell cycle stage and how each cell was cut.

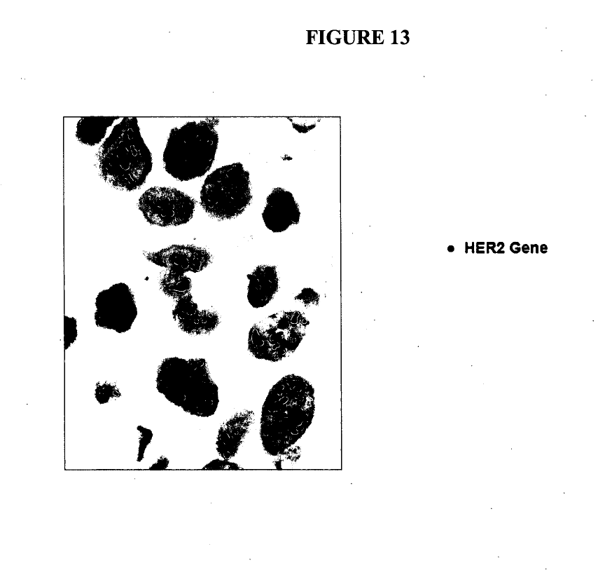

FIG. 13 is a photomicrograph at 100× showing dots of deposited silver metal as clusters of HER2 (due to gene amplification) gene in a 5 μm-section of formalin-fixed, paraffin-embedded BT-474 (human breast ductal carcinoma cell line) xenograft tumor. As can readily be seen, there are clusters of multiple copies of HER2 gene signals in each cell.

### Example 3

### Co-Detection of HER2 and Chromosome 17 Centromere in Tissue with Sequential Hybridization Steps

In this example, the method of the present invention was used to sensitively and selectively detect copy numbers of.the HER2 gene in combination with a Chromosome 17 centromere (CEP 17) label in situ, in formalin-fixed, paraffin-embedded xenograft tumor tissue sections using a double stranded DNA probe (HER2) and a single stranded oligoprobe (CEP 17) with different stringency characteristics. Inclusion of CEP 17 detection allows for the relative copy number of the HER2 gene to be determined. For example, normal samples will have a HER2/CEP 17 ratio of less than 2, whereas samples in which the HER2 gene is reduplicated will have a HER2/CEP 17 ratio of greater than 2.0.

The assay was completely automated on a Ventana BenchMark™ XT. The slides with the fixed cells were baked/heated at 65° C. for 20 minutes and deparaffinized using EZ Prep (Ventana P/N 950-102). The slides were further processed for heat pretreatment using Reaction Buffer (Ventana P/N 950-300) and Protease 3 (Ventana P/N 760-2020) prior to hybridization step.

Detection of HER2 genes was performed as follows. DNP-labeled HER2 probe (Ventana P/N 780-4332) was hybridized at 52° C. for 2 hours after co-denaturing the target and the probe on the slides at 95° C. for 12 minutes. Stringency wash steps were conducted using SSC (Ventana P/N 950-110). Successively, an anti-DNP rabbit polyclonal antibody (Ventana P/N 780-4335), then a goat anti-rabbit-Horseradish Peroxidase conjugated antibody (a component of ultraVIEW™ SISH Kit (Ventana P/N 780-001) was incubated with the tissue. Slides were washed with an un-buffered solution containing a surfactant and water SISH Wash (Ventana P/N 780-002). Approximately 100 μl of the wash remained on the slide prior to reagent application. One hundred (100) μl each of Silver Chromogen A (0.36% silver acetate), Silver Chromogen B (0.18% hydroquinone), and Silver Chromogen C (0.09% hydrogen peroxide) (components of ultraVIEW™ SISH Kit Ventana P/N 780-001) were dispensed onto the slide. Silver Chromogen A was applied first and incubated for 4 minutes at 37° C. Without rinsing, Silver Chromogen B was applied to the slide and allowed to incubate an additional 4 minutes at 37° C. Finally, Silver Chromogen C was added to the pool of Silver Chromogen A and B and incubated for a final 12 minutes at 37° C. Prior to detecting CEP 17, the slides were washed with 2×SSC.

Next, detection of CEP 17 sequences was performed as follows. DNP-labeled chromosome 17 centromere oligoprobe (Ventana P/N 780-4331) was hybridized at 44° C. for 1 hour after co-denaturing the target and the probe on the slides at 95° C. for 12 minutes. Then, colorimetric detection of chromosome 17 centromere was performed using anti-DNP rabbit polyclonal antibody (Ventana P/N 780-4335), UltraMap™ anti-rabbit alkaline-phosphatase conjugated antibody (Ventana P/N 760-4314), and fast red-naphthol phosphate substrate (components of ultraVIEW™ Universal Alkaline Phosphatase Red Detection Kit, Ventana PN 760-501). All tissue sections were counterstained with Hematoxylin II (Ventana PN 790-2208) and Bluing Reagent (Ventana PN 760-2037).

FIG. 14 is a photomicrograph at 100× showing dots of deposited silver metal as discrete single copies of HER2 gene in a 5 μm-section of formalin-fixed, paraffin-embedded MCF7 (A highly rearranged, near triploid human breast carcinoma cell line) xenograft tumor. There are one to three signals per cell, dependent on the cell cycle stage and how each cell was cut. Also shown are red dots resulting from fast red naphthol staining indicating single copies of CEP 17. As with the HER2 gene, there are one to three signals per cell, the normal complement. Thus, the results demonstrate a HER2/CEP 17 ratio of less than 2 can readily be detected using the method of the present invention.

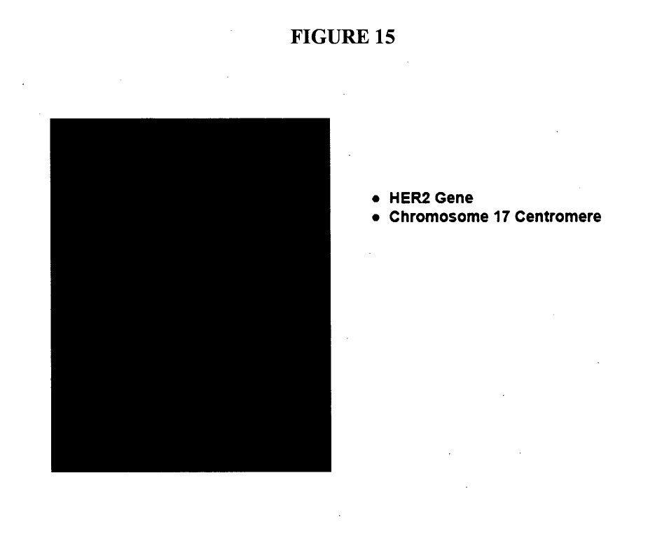

FIG. 15 is a photomicrograph at 100× showing dots of deposited silver metal as discrete single copies of HER2 gene in a 5 μm-section of formalin-fixed, paraffin-embedded ZR-75-1 (A highly rearranged, near triploid human breast carcinoma cell line) xenograft tumor. Also shown are red dots resulting from fast red naphthol staining indicating single copies of CEP 17. As with the HER2 gene, there are one to three signals per cell, dependent on the cell cycle stage and how each cell was cut within a tissue section.

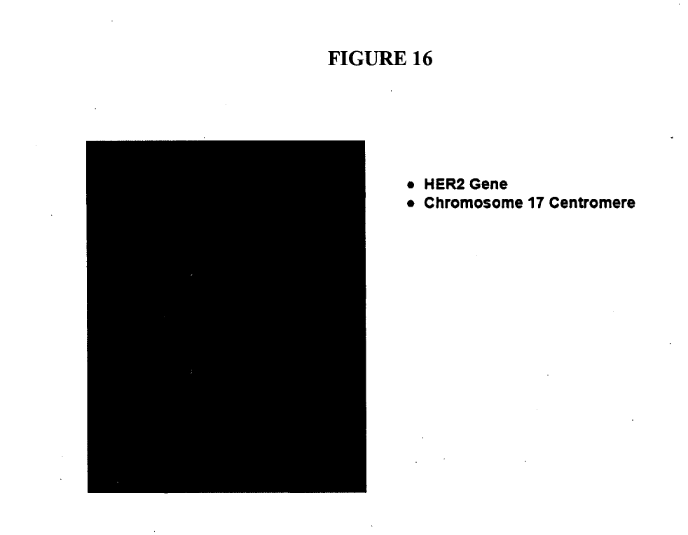

In comparison, FIG. 16 is a photomicrograph at 100× showing dots of deposited silver metal as clusters of HER2 gene (due to amplification of copy number) in a 5 μm-section of formalin-fixed, paraffin-embedded BT-474 (human breast ductal carcinoma cell line) xenograft tumor. Also shown are red dots resulting from fast red naphthol staining indicating single copies of CEP 17. As can readily be seen, there are amplified HER2 gene signals in each cell, but only a few CEP 17 signals per cell.

### Example 4

### Co-detection of HER2 gene and Chromosome 17 Centromere in Tissue with Co-Hybridization Step

In this example, the method of the present invention was used to sensitively and selectively detect copy numbers of the HER2 gene in combination with a Chromosome 17 centromere (CEP 17) label in situ, in formalin-fixed, paraffin-embedded xenograft tumor tissue sections using two double-stranded DNA probes. HER2 probe is used in combination with a CEP 17 probe that hybridizes to the alpha satellite DNA located at the centromere of chromosome 17 (17p11.1-q11.1). Inclusion of the CEP 17 probe allows for the relative copy number of the HER2 gene to be determined. For example, normal samples will have a HER2/CEP17 ratio of less than 2, whereas samples in which the HER2 gene is reduplicated will have a HER2/CEP17 ratio of greater than 2.0.

Detection of the HER2 genes by silver deposition was performed essentially as described in the previous example, except the HER2 probe was constructed by dinitrophenol (DNP) labeling a composite repeat depleted (CORD) probe. CORD probes are described in detail in U.S. Provisional Pat. App. Ser. No. 60/841,896, filed Sep. 1, 2006, incorporated in its entirety herein by reference. Briefly, CORD probes are completely or substantially completely depleted of repetitive nucleic acid elements (e.g. Alu repeats, LI repeats and Alpha satellite DNA), and correspond on a segment by segment basis to unique coding or non-coding elements of the target sequence.

Unique sequence elements throughout a 500,000 base pair region of chromosome 17 that includes the HER2 gene were identified and amplified by PCR. Repetitive sequences were identified and amplification primers were then selected to amplify non-repeat sequences. Oligonucleotide primers were selected for a Tm as close as possible to 69° C., and a position as close as possible to each end of the unique sequence segment, to maximize the size of the PCR products. Forward primers were synthesized with a 5′ phosphate, whereas reverse primers were not. The resulting amplification products possessed 5′ phosphates at a single end.

The resulting fragments were processed and ligated together as a mixture without regard to order or orientation. The ligated material was amplified stepwise by random priming amplification using Phi29 DNA polymerase. The HER2 probe DNA was labeled with DNP, using the MIRUS kit according to manufacturer's instructions (P/N MIR 3800; Mirus Bio Corp. Madison, Wis.).

The Chromosome 17 centromere probe was developed from plasmid pYAM7-29 (ATCC number 65442) containing copies of a microsatellite repeat sequence in an approximately 2700 base pair insert cloned in pUC19. The sequence of approximately 1000 base pairs from each end of the plasmid was determined using primers M13 F and M13 R as shown in Table 1. The plasmid was labeled with fluorescein using the MIRUS kit according to manufacturer's instructions.

The assay was completely automated on a Ventana BenchMark™ XT. The slides were baked/heated at 65° C. for 20 minutes and deparaffinized using EZ Prep (Ventana P/N 950-102). Then, slides were subjected for heat pretreatment using CC1 (Ventana P/N 950-124) and fixed using formaline-based fixative RiboFix™, a component of RiboMap™ (Ventana P/N 760-102). The slides were further processed for another heat pretreatment using Reaction Buffer (Ventana P/N 950-300) and Protease 3 (Ventana P/N 760-2020) prior to hybridization step. DNP-labeled double-stranded HER2 probe and fluorescein-labeled double-stranded chromosome 17 centromere probe were co-hybridized at 52° C. for 2 hours after co-denaturing the targets and the probes on the slides at 95° C. for 12 minutes. Stringency wash steps were conducted using SSC (Ventana P/N 950-110). Successively, an anti-DNP rabbit polyclonal antibody (Venatana P/N 780-4335), then a goat anti-rabbit-Horseradish Peroxidase conjugate antibody (a component of ultraVEW™ SISH Kit, Ventana P/N 780-001) was incubated with the tissue. Prior to application of chromogenic reagents, slides were washed with an un-buffered solution containing a surfactant and water (SISH Wash (Ventana P/N 780-002). Appoximately 100 μl of the wash remained on the slide prior to reagent application. One hundred II each of Silver A (0.36% silver acetate), Silver B (0.18% hydroquinone), and Silver C (0.09% hydrogen peroxide) (components of ultraVIEW™ SISH Kit, Ventana P/N 780-001), were dispensed onto the slide. Silver A is applied first and incubated for 4 minutes at 37° C. Without rinsing, Silver B is applied to the slide and allowed to incubate an additional 4 minutes at 37° C. Finally, Silver C is added to the pool of Silver A and B and incubated for a final 12 minutes at 37° C.

Colorimetric detection of chromosome 17 centromere was performed using mouse anti-fluorescein antibody (a component of ISH iVIEW™ Blue Detection Kit, Ventana P/N 760-092), rabbit anti-mouse (Amplifier A of Amplification Kit, Ventana P/N 760-080), UltraMap™ anti-rabbit alkaline-phosphatase conjugated antibody (Ventana P/N 760-4314), and fast red-naphthol phosphate substrate (components of ultraVIEW™ Universal Alkaline Phosphatase Red Detection Kit, Ventana PN 760-501). All tissue sections were counterstained with Hematoxylin II (Ventana PN 790-2208) and Bluing Reagent (Ventana PN 760-2037) as described in the previous example.

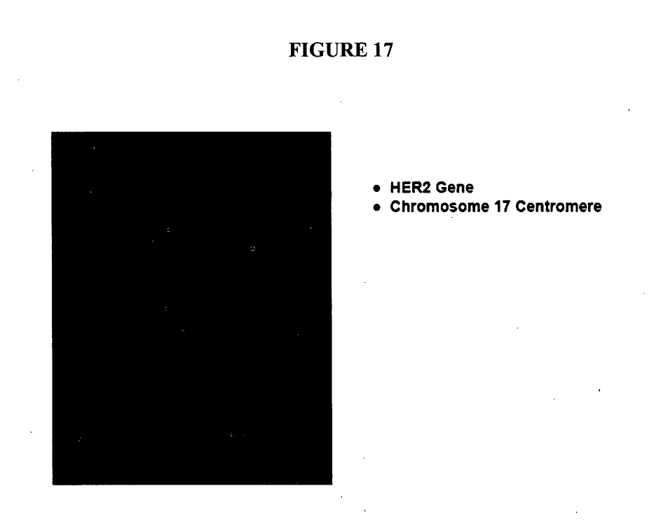

FIG. 17 is a photomicrograph at 100× showing dots of deposited silver metal as discrete single copies of HER2 gene in a 5 μm-section of formalin-fixed, paraffin-embedded MCF7 (a highly rearranged, near triploid human breast carcinoma cell line) xenograft tumor. There are one to three signals per cell, dependent on the cell cycle stage and how each cell was cut. Also shown are red dots resulting from fast red naphthol staining indicating single copies of CEP 17. As with the HER2 gene, there are one to three signals per cell, the normal complement. Thus, the results demonstrate a HER2/CEP 17 ratio of less than 2 can readily be detected using the method of the present invention.

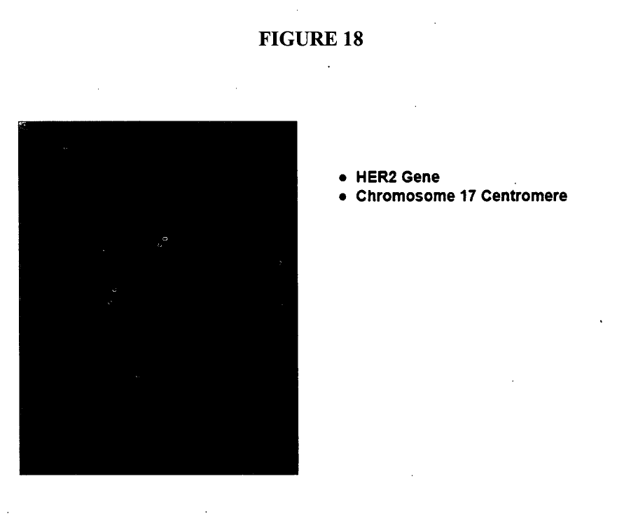

FIG. 18 is a photomicrograph at 100× showing dots of deposited silver metal as discrete single copies of HER2 gene in a 5 μm-section of formalin-fixed, paraffin-embedded ZR-75-1 (a highly rearranged, near triploid human breast carcinoma cell line) xenograft tumor. Also shown are red dots resulting from fast red naphthol staining indicating single copies of CEP 17. As with the HER2 gene, there are one to three signals per cell, dependent on the cell cycle stage and how each cell was cut within a tissue section.

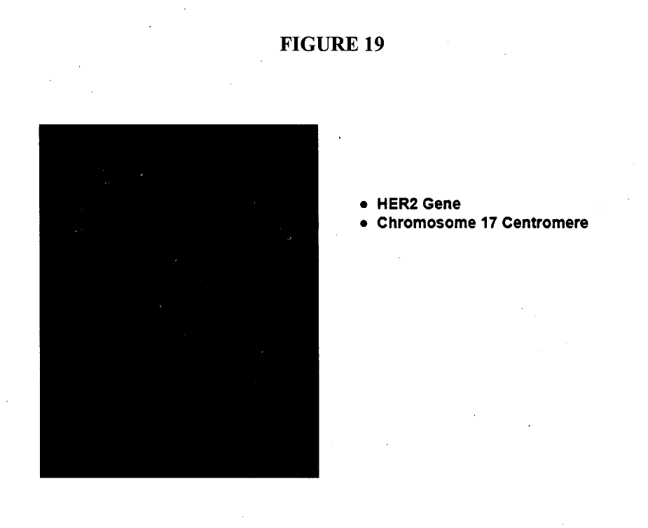

In comparison, FIG. 19 is a photomicrograph at 100× showing dots of deposited silver metal as clusters of HER2 genes (due to amplification of copy number) in a 5 μm-section of formalin-fixed, paraffin-embedded BT474 (human breast ductal carcinoma) xenograft tumor. Also shown are red dots resulting from fast red naphthol staining indicating single copies of CEP 17. As can readily be seen, there are amplified HER2 gene signals in each cell, but only a few CEP 17 signals per cell. Thus, the results demonstrate a HER2/CEP 17 ratio of more than 2 can readily be detected using the method of the present invention.

TABLE 1

| | |
|---|---|
| Primer | Sequence |

| | |
|---|---|
| M13 F | CGCGACGGGGACTCTAGAGTCGACCTGCAGAATCT |
| (SEQ ID NO. 1) | GCAAGTGCATATTTGGACCTCTGTGAGGaATTCGt |
| | TGGAAACGGGATAATTTCAGCTGACTAAACAGAANC |
| | AGTCTCAGAATCTTCTTTGTGATGTTTGCATTCACA |
| | TCCCCGAGTTGAACTTTCCTTTCAAAGTTCACGTTT |
| | GAAACACTCTTTTTGCAGGATCTACAAGTGGATATT |
| | TGGACCACTCTGTGTCCTTCGTTCGAAACGGGTATA |
| | TCTTCACATGACATCTAGACAGAAGCATCCTCAGAA |
| | GCTTCTCTGTGATGACTGCATTCAACTCACGGAGTT |
| | GAACACTCCTTTTGAGAGCGCAGTTTTGAAACTCTC |
| | TTTCTGTGGCATCTGCAAGGGGACATGTAGACCTCT |
| | TTGAAGATTTCGTTGGAAACGGAATCATCTTCACAT |
| | AAAAACTATACAGATGCATTCTCAGGAACTTTTTGG |
| | TGATGTTTGTATTCAACTCCCAGAGTTGAACTTTCC |
| | TTTGGAAAGAGCAGCTATGAAACACTCTTTTTCTAG |
| | AATCTGCAAGTGGACGTTTGGAGGGCTTTGTGGTTT |
| | GTGGTGGAAAAGGAAATATCTTCACCTCAATACTAG |
| | ATAGAAGCATTCTCAGAAACTGCTTTGTGATGATTG |
| | CATTCACCTCACAGAGTTGAACATTCCTATTGATAG |
| | AGCAGNTTGGAAACACTCTTGTTGTGGAATGTGCAA |
| | GTGGAGATTTGGAGCGCTTTGAGGCCTATGGTAGTA |
| | AAGGGAATAGCTTCATAGAAAAACTAGACAGAAGCA |
| | TTCTCAGAAAATACTTTGTGATGATTGAGTTNAACT |
| | CACAGAGCTGAACATTCCTTTGGATGGAGCAGGTTN |
| | GAGACACACTTTTTGTAGAATCTACAAGTGGATATT |
| | TGGACCTCTCTGANGGATTTCGTTGGAAACGGGATA |
| | ACTGCACCTAACTAAACGGAAGCATTCCTCAGAAAC |
| | TTCTNGGTGATGTTTGCATTCAAATCCCANAGTNGA |
| | ACCTTCCTTTGAAAGTNCAGGGTTGAANNCCNCTTT |
| | TTGTAGGGATCTGCANGTGGATNTTGGGACCCTCTG |
| | NGGCCTTCGTTCGAAACGGGTATATCTTTCCCANNA |
| | AATNTAAACAGAAGCCTTCCCCAAANCTCCCCGGGN |
| | AGATGCCNNCNCNCAANAGNNNANCCCCCNTTGGAA |
| | GANGCAGGTGAAACCCTCTTTTGGGAANNCNCAAGG |
| | GAATTNGNNCCCCCCCAAANGTCTTGGAANNGGAAT |
| | TTCCANAAAANAANANANCTTCCAAAACTCCCGGGG |
| | NTNGGNNACCCNANTNCNNTGTTTTNAAAATTNAAA |
| | NNNTTTTCAGGGCCAAGGGAATTGGGCTCCNGCGGG |
| | GGGAAAATTGGCCCAAANGGGANNNNCCAACNTTGG |
| | NGTCCCCAAGAGGNCCTNNAAANCCCCTTGGGCNGG |
| | GNTCCTTATTNGGNNAAACNAANAANN |
| M13 R | CCACTGCTGCCTGCGAAGTGTGTTTCTAAACTGCT |
| (SEQ ID NO. 2) | ACATCGCAAGGAATGCTCAGCTCTGTGAGTTCAAC |
| | TCAATCATCCCAAAGAATTTTCTGAGAAAGCTTCTG |
| | NCTTCTTTTTATAGGAAGTTATTTCCTTTACTACGG |
| | TACTCCTCAAAGAGTGCAATGATCCCTTGCAGTTTC |
| | TACAAAAAGAGTGTTTCAAACCTGAACTATCAAAGA |
| | AAGGTTCCACACTGTGAGTTGAATGCAGACATCACG |
| | AAGAAGGTTCTGAGAATGCTTCTGTTTAGTTCTGTG |
| | CAGTTTATCCCGTTTCCAACGAAATCCTCAGAGAGG |
| | ACCAAATATCCACTTGCAGTTTCTACAAAAAGAGTG |
| | TTTCAAAGCTGAACTATCAAAGAAAGGTTCAGCACT |
| | GTGAGTTGAATGCAAACATCACGAAGAGGGTTCTGA |
| | GAATGCTTCTGTTTTAGTTCTGTGCGGGTTATCCCG |
| | TTTCCAACGAAATCCTCAGAGCGGTCCAAATATCTA |
| | CTTGCAGTTTCTACAGAAAGACCGTTTCAAACCTGA |
| | ACTATCAAAGAAAGGTTCAACACTGTGAGTTGAATG |
| | CAAACATCACGAAGAAGGTTCTGAGAATGCTTCTGT |
| | TTAGTTCTGTGCGGTTTATCCCGTTTCCAACGAAAT |
| | CCTCAGAGAGGCCTAAATATCCACTTGCACATTCTA |
| | CAAATAGTGTGNTTCGAAACTGCTCCATCCAAAGGA |
| | ATGTTCAGCTCTGTGAGTTAAACTCAGTCGTCACCA |
| | AGAGTTTTCTGTGAATGCTACTGTCTAGCTTTTATA |
| | TGAAGCTATTTCCTTTACTACCATAGGCCTCAAAGC |
| | GGTCCCATATCTCCACTTGCAGATTCTACACAAAAG |
| | AGAGTTTCCAAACTGCTCTGTCAAAGGGAATGTTCA |
| | ACTCTGTGACTGGAATGCAATCATCACNAAAGTAGT |
| | TTCTGAGANTGCTNCTATCTAGCTTTTACGGGAAGA |
| | TAATTCCTTTTCCACCACANGGCCTCAANGCCCTCN |
| | AATGTCCNCTTGCCAGATTCTGGAAAAAGAGNGTTT |
| | CAAGCTTCTCTCTCGAAAGGNANGTTCACCTNGGGG |
| | GTTGAANGCAGCCTCCCAANAAGTTTCTGAAAAGCT |
| | NCGGTTAGCTTNCCGGGANATTCNCCGTTCCACGAA |
| | NTTTCCAAANGGNCCAAANTCCCTTGCAATCCCCNA |
| | AAGANGATGGGANCGCNTTTGAAAAGAACNTCACNN |
| | GGGGGTGGAAGNANCNCCAAAANTTNCGANNGGCTT |
| | CCCCCNGTTTNTGGACANATTTTTTCCCNNGNCNGA |
| | NNCCCAAGGNCCTTTGAANCNCCAANGGGTTCAANC |
| | CCTTAAAAAGGGACTNGGGGTAACCCCCNCNAATTC |
| | GACCTTTTTTTTTGAATTCCTTCAACNNGGGGCNCT |
| | CCTNTCCAAAATTNCCCCNANGGCCTNGGAAACNAA |
| | NTGGNTTTTTTAANTTAACGNNNNTCNNTCCTNN |

### Example 5

### Co-Detection of HER2 Protein, HER2 Gene and Chromosome 17 Centromere in Tissue

In this example, the method of the present invention was used to sensitively and selectively detect copy numbers of the HER2 gene in combination with a chromosome 17 centromere (CEP 17) label in situ, and in addition detect HER2 protein on the same sample/slide, in breast cancer cell line xenograft tumors. HER2 probe is used in combination with a CEP 17 probe that hybridizes to the alpha satellite DNA located at the centromere of chromosome 17 (17p11.1-q11.1). Inclusion of the CEP17 probe allows for the relative copy number of the HER2 gene to be determined. For example, normal HER2 gene samples will have a HER2/CEP17 ratio of less than 2, whereas samples in which the HER2 gene is amplified will have a HER2/CEP17 ratio of greater than 2.0. HER2 protein is detected using a rabbit monoclonal antibody clone 4B5 using BCIP/NBT chromogen. This triple detection combination with preservation of morphology on a single sample slide is a major advance in the art given the complexities of balancing hybridization conditions for the two probes and the immunohistochemistry (IHC) conditions for the detection of the HER2 protein. A schematic representing the protocol used in this example is shown in FIG. 20.

Detection of the HER2 genes by silver deposition was performed essentially as described in Example 4. Detection of CEP 17 was also performed essentially as described in Example 4.

The assay was completely automated on a Ventana BenchMark™ XT. The slides were baked/heated at 65° C. for 20 minutes and deparaffinized using EZ Prep (Ventana P/N 950-102). Then, slides were subjected to heat pretreatment using CC1 (Ventana P/N 950-124) prior to conducting HER2 protein immunohistochemical staining. Tissue sections were incubated with rabbit monoclonal anti-HER2 antibody, clone 4B5 (Ventana P/N 790-2991), UltraMap anti-rabbit alkaline phosphatase-labeled antibody (Ventana P/N 760-4314), and BCIP-NBT substrate (components of ISH iVIEW Blue Detection Kit, Ventana P/N 760-092).

After HER2 protein immunohistochemistry, the tissue sections were fixed using formaline-based fixative RiboFix, a component of RiboMap (Ventana P/N 760-102). The slides were further processed for another heat pretreatment using Reaction Buffer (Ventana P/N 950-300) and Protease 3 (Ventana P/N 760-2020) prior to the hybridization step.

Silver deposition detection of HER2 genes was performed as follows. DNP-labeled HER2 probe and fluorescein-labeled chromosome 17 centromere probe were co-hybridized at 52° C. for 2 hours after co-denaturing the targets and the probes on the slides at 95° C. for 12 minutes. Stringency wash steps were conducted using SSC (Ventana P/N 950-110). Successively, an anti-DNP rabbit polyclonal antibody (Ventana P/N 780-4335), then a goat anti-rabbit-Horseradish Peroxidase conjugated antibody (a component of ultraVIEW™ SISH Kit, Ventana PiN 780-001) was incubated with the tissue. Prior to application of chromogenic reagents, slides were washed with an un-buffered solution containing a surfactant and water (SISH Wash, Ventana P/N 780-002). Approximately 100 μl of the wash remained on the slide prior to reagent application. One hundred μl each of Silver A (0.36% silver acetate), Silver B (0.18% hydroquinone), and Silver C (0.09% hydrogen peroxide) (components of ultraVIEW™ SISH Kit P/N 780-001), were dispensed onto the slide. Silver A is applied first and incubated for 4 minutes at 37° C. Without rinsing, Silver B is applied to the slide and allowed to incubate an additional 4 minutes at 37° C. Finally, Silver C is added to the pool of Silver A and B and incubated for a final 12 minutes at 37° C.

Colorimetric detection of chromosome 17 centromere was performed using mouse anti-fluorescein antibody (a component of ISH iVIEW™ Blue Detection Kit, Ventana P/N 760-092), rabbit anti-mouse (Amplifier A of Amplification Kit, Ventana P/N 760-080), UltraMap™ anti-rabbit alkaline-phosphatase conjugated antibody (Ventana P/N 760-4314), and fast red-naphthol phosphate substrate (components of ultraVIEW™ Universal Alkaline Phosphatase Red Detection Kit, Ventana PN 760-501). All tissue sections were counterstained with Hematoxylin II (Ventana PN 790-2208) and Bluing Reagent (Ventana PN 760-2037) as described in the previous example.

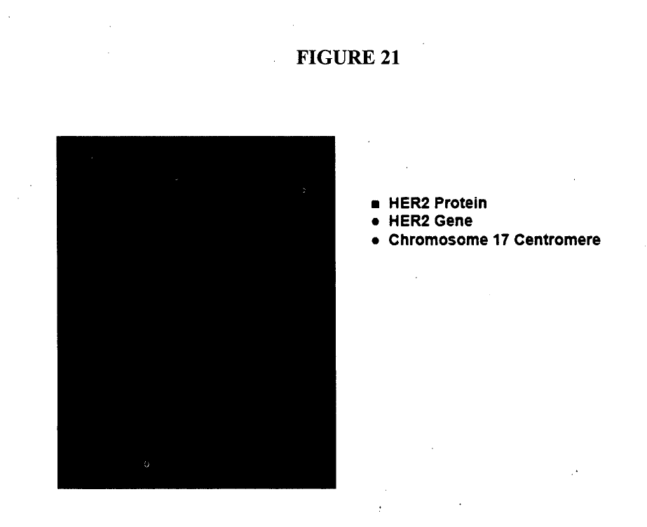

FIG. 21 is a photomicrograph at 100× showing dots of deposited silver metal as discrete single copies of HER2 gene, red dots of fast red naphthol staining as chromosome 17 centromeres in a 5 μm-section of formalin-fixed, paraffin-embedded MCF7 (A highly rearranged, near triploid human breast carcinoma cell line) xenograft tumor. MCF7 xenograft tumors are known not to express detectable HER2 protein (Level 0). As it was expected, no HER2 protein was visualized in MCF7 tumor sections.

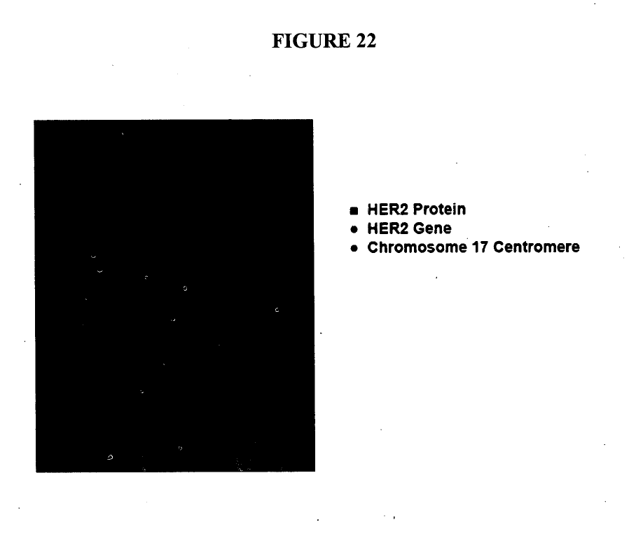

FIG. 22 is a photomicrograph at 100× showing dots of deposited silver metal as discrete single copies of HER2 gene, red dots of fast red naphthol staining as chromosome 17 centromeres, and blue/purple staining of limited amount of HER2 in a 5 μm-section of formalin-fixed, paraffin-embedded ZR-75-1 (A highly rearranged, near triploid human breast carcinoma cell line) xenograft tumor. ZR-75-1 xenograft tumor is known to express “Level 1” of HER2 protein.

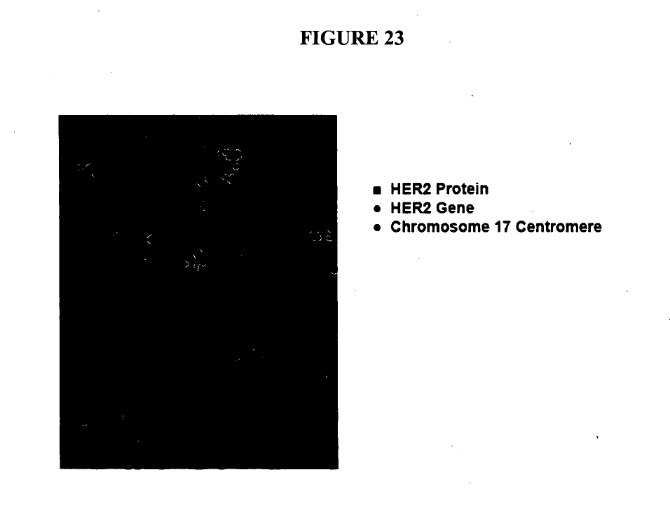

In comparison, FIG. 23 is a photomicrograph at 100× showing dots of deposited silver metal as clusters of HER2 gene, red dots of fast red naphthol staining as chromosome 17 centromeres, and blue/purple staining of amplified amount of HER2 in a 5 μm-section of formalin-fixed, paraffin-embedded BT-474 (human breast ductal carcinoma cell line) xenograft tumor. BT-474 is known to express amplified HER2 protein “Level 3”. Amplified HER2 protein is visualized as dark blue/purple in. the cell membrane.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for detecting a plurality of target molecules in a test sample, comprising:
detecting a first target molecule of said plurality of target molecules by i) binding an enzyme to the first target molecule, ii) contacting the enzyme with metal ions in the presence of an oxidizing agent and a reducing agent, whereby the metal ions are reduced to elemental metal, thereby depositing the elemental metal in the vicinity of the enzyme, and iii) determining the presence, amount or level of the deposited metal in the vicinity of the enzyme bound to the first target molecule; and
detecting at least one second different target molecule of said plurality of target molecules in the test sample by generating a detectable signal at the site of the second target molecule that is distinguishable from said deposited metal.

2. The method of claim 1, wherein at least some portion of labeling of said first target molecule and at least some portion of labeling of said at least one second different target molecule occurs simultaneously.

3. The method of claim 1, wherein the metal ions are selected from the group consisting of silver, gold, iron, mercury, nickel, copper, platinum, palladium, cobalt, iridium ions and mixtures thereof.

4. The method of claim 1, wherein the metal ions are silver ions.

5. The method of claim 1, wherein the enzyme is an oxido-reductase.

6. The method of claim 1, wherein the enzyme is peroxidase.

7. The method of claim 1, wherein the enzyme is horseradish peroxidase.

8. The method of claim 1, wherein the enzyme is conjugated to avidin or streptavidin.

9. The method of claim 1, wherein the enzyme is conjugated to an antibody.

10. The method of claim 9, wherein said antibody is an anti-first target molecule antibody.

11. The method of claim 1, wherein the oxidizing agent is an oxygen-containing oxidizing agent.

12. The method of claim 1, wherein the reducing agent is selected from the group consisting of hydroquinone, a hydroquinone derivative, n-propyl gallate, 4-methylaminophenol sulfate, 1,4 phenylenediamine, o-phenylenediamine, chloroquinone, bromoquinone, 2-methoxyhydroquinone, hydrazine, 1-phenyl-3-pyrazolidinone and dithionite salts.

13. The method of claim 1, wherein detection of said second different target molecule is performed using one selected from the group consisting of a radioactive label, a colorimetric label, a fluorescent label, and a chemiluminescent label.

14. The method of claim 13, wherein the substance is conjugated to an antibody.

15. The method of claim 13, wherein the detection of said second different target molecule includes binding an enzyme to the second target molecule via a primary antibody that specifically binds to the second different target molecule, and a secondary antibody that is conjugated with the enzyme and binds to the primary antibody.

16. The method of claim 13, wherein an enzyme facilitates detection of the presence of the detectable substance.

17. The method of claim 1, wherein detecting said second different target molecule includes binding an enzyme to the second different target molecule via a nucleic acid probe that specifically hybridizes to the second different target molecule and is labeled with a detectable marker.

18. The method of claim 17, wherein the enzyme binds to the detectable marker via an antibody that specifically binds to the detectable marker.

19. The method of claim 17, wherein the detectable marker is biotin, dinitrophenyl, a radio-isotope or a fluorescent label.

20. The method of claim 19, wherein the fluorescent label is selected from the group consisting of fluorescein isothiocyanate (FITC), Texas Red, rhodamine and Cy5.

21. The method of claim 17, wherein the enzyme binds to the detectable marker via a primary antibody that specifically binds to the detectable marker, and a secondary antibody that is conjugated with the enzyme and binds to the primary antibody.

22. The method of claim 1, wherein the second different target molecule is a polynucleotide sequence and the detection of said polynucleotide sequence includes binding an enzyme to the polynucleotide sequence via a nucleic acid probe that specifically hybridizes to the polynucleotide sequence and is labeled with a detectable marker.

23. The method of claim 1, wherein the second different target molecule is a polynucleotide sequence.

24. The method of claim 23, wherein said polynucleotide sequence is a gene, a gene product, a non-coding sequence, or a genome.

25. The method of claim 23, wherein the second different target molecule is HER2/neu gene or gene product, or a Chromosome 17 centromere sequence.

26. The method of claim 1, further comprising:
comparing the presence, amount or level of the substance used to detect the second different target molecule with the presence, amount or level of deposited metal;
optionally comparing the presence, amount or level of the substance used to detect the second different target molecule with that of a reference sample;
optionally comparing the presence, amount or level of deposited metal with that of a reference sample; and
determining a disease status of a patient from whom the test sample is derived.

27. The method of claim 26, wherein the reference sample comprises cells or tissue from a normal, healthy individual.

28. The method of claim 26, wherein the disease status is disease determination or classification, prognosis, drug efficacy, patient responsiveness to therapy, whether adjuvant or combination therapy is recommended, or likelihood of recurrence of disease.

29. The method of claim 26, wherein the disease is selected from the group consisting of benign tumors, cancer, hematological disorders, autoimmune diseases, inflammatory diseases, cardiovascular diseases, nerve degenerative diseases and diabetes.

30. The method of claim 26, wherein the disease status is patient response to therapy.

31. The method of claim 1, further comprising:
detecting at least a third different target molecule of said plurality of target molecules in the test sample by generating a detectable signal at the site of the third target molecule that is distinguishable from said deposited metal and distinguishable from the detectable signal at the site of the second target molecule.

32. The method of claim 31, wherein detection of said third target molecule is performed using one selected from the group consisting of a radioactive label, a colorimetric label, a fluorescent label, and a chemiluminescent label.

33. The method of claim 32, wherein the label is conjugated to an antibody.

34. The method of claim 32, wherein an enzyme facilitates detection of the presence of the detectable substance.

35. The method of claim 31, wherein the detection of the third target molecule includes binding an enzyme to the third target molecule via a primary antibody that specifically binds to third target molecule, and a secondary antibody that is conjugated with the enzyme and binds to the primary antibody.

36. The method of claim 31, further comprising:
comparing the presence, amount or level of the substance used to detect the third target molecule with that of a reference sample; and
determining a disease status of a patient from whom the test sample is derived.

37. The method of claim 30, further comprising:
comparing the presence, amount or level of the substance used to detect the third different target molecule with the presence, amount or level of deposited metal; and
determining a disease status of a patient from whom the test sample is derived.

38. The method of claim 37, further comprising:
comparing the presence, amount or level of the substance used to detect the third different target molecule with the presence, amount or level of the substance used to detect the second target molecule; and
determining a disease status of a patient from whom the test sample is derived.

39. The method of claim 31, further comprising: comparing the presence, amount or level of the substance used to detect the third different target molecule with the presence, amount or level of the substance used to detect the second target molecule; and
determining a disease status of a patient from whom the test sample is derived.

40. The method of claim 31, wherein the target molecules are predetermined.

41. The method of claim 40, wherein the first target molecule is HER2/neu gene, the second target molecule is HER2 protein, and the third target molecule is a chromosome 17 centromere sequence.

42. The method of claim 31, wherein the target molecules are spaced in a predetermined geometric pattern.

43. The method of claim 31, wherein the signal generated by detection of said third target molecule is selected from the group consisting of radioactive signal, calorimetric signal, fluorescent signal, and chemiluminescent signal.

44. The method of claim 43, wherein said signal generated by binding a label to said third target molecule is distinguishable from said metal deposition and from said signal generated by the label bound to said second target molecule by one of the group consisting of different fluorescent color, different brightfield color, different radioactive emission, and different chemiluminescent color.

45. The method of claim 1, wherein the target molecules are predetermined.

46. The method of claim 45, wherein the target molecules are selected from the group consisting of HER2 gene, HER2 protein, and chromosome 17 centromere sequence.

47. The method of claim 1, wherein the target molecules are selected from the group consisting of receptor for fibrin, receptors for VEGF, Flt4, receptor for VEGF-165, Tie1, Tie2, receptor for ephrine A1-5, receptor for ephrine B1-5, epidermal growth factor receptors (EGFR), platelet-derived growth factor receptors (PDGFR), nerve growth factor receptors (NGFR), HER1, HER2/neu, HER3, HER4, Kit, c-Kit, Src, Fes, JAK, Fak, Btk, Syk/ZAP-70, and Abl.

48. The method of claim 1, wherein the signals from the first and second target molecules are spaced in a predetermined geometric pattern.

49. The method of claim 1, wherein the signal generated by binding a label to said second target molecule is selected from the group consisting of radioactive signal, colorimetric signal, fluorescent signal, and chemiluminescent signal.

50. The method of claim 48, wherein said signal generated by binding a label to said second target molecule is detected.

51. A kit for detection of multiple targets in a sample, comprising:
i) metal ions selected from the group consisting of silver, gold, iron, mercury, nickel, copper, platinum, palladium, cobalt, iridium ions and a mixture thereof;
ii) an oxidizing agent;
iii) a reducing agent, and
iv) at least two binding moieties that bind to two different target molecules in a sample.

52. The kit of claim 51, wherein the weight ratio of the metal ions to the reducing agent ranges from 1:5 to 5:1, and the weight ratio of the reducing agent to the oxidizing agent ranges from 1:10 to 10:1.

53. The kit of claim 50, wherein the binding moieties are selected from the group consisting of antibody, antibody fragments, peptide, nucleic acids, nucleic acid probes, carbohydrates, drugs, steroids, products from plants, animals, humans and bacteria, and synthetic molecules, wherein each member has an affinity for binding to the target molecule.

54. The kit of claim 53, wherein at least one target molecule is a target gene, non-coding sequence, or genome and the binding moiety is a nucleic acid probe that binds to the target gene or genome.

55. The kit of claim 54, further comprising an enzyme.

56. The kit of claim 54, wherein the enzyme is associated with the target molecule either via a primary antibody that binds to the target molecule, or via a primary antibody that binds to the binding moiety.

57. The kit of claim 56, wherein the enzyme is a peroxidase; the metal ions are silver ion; the oxidizing agent is hydrogen peroxide; and the reducing agent is hydroquinone.

58. The kit of claim 51, further comprising: instruction for performing the detection using the kit.


SEQUENCE LISTING (2 sequences)

SEQ ID NO: 1 - DNA, 1465 bp, Homo sapien
  misc_feature (105)..(105): n is a, c, g, or t
cgcgacggggactctagagtcgacctgcagaatctgcaagtgcatatttggacctctgtgaggaattcgttggaaacgggataatttcagctgactaaacagaancagtctcagaatcttctttgtgatgtttgcattcacatccccgagttgaactttcctttcaaagttcacgtttgaaacactctttttgcaggatctacaagtggatatttggaccactctgtgtccttcgttcgaaacgggtatatcttcacatgacatctagacagaagcatcctcagaagcttctctgtgatgactgcattcaactcacggagttgaacactccttttgagagcgcagttttgaaactctctttctgtggcatctgcaaggggacatgtagacctctttgaagatttcgttggaaacggaatcatcttcacataaaaactatacagatgcattctcaggaactttttggtgatgtttgtattcaactcccagagttgaactttcctttggaaagagcagctatgaaacactctttttctagaatctgcaagtggacgtttggagggctttgtggtttgtggtggaaaaggaaatatcttcacctcaatactagatagaagcattctcagaaactgctttgtgatgattgcattcacctcacagagttgaacattcctattgatagagcagnttggaaacactcttgttgtggaatgtgcaagtggagatttggagcgctttgaggcctatggtagtaaagggaatagcttcatagaaaaactagacagaagcattctcagaaaatactttgtgatgattgagttnaactcacagagctgaacattcctttggatggagcaggttngagacacactttttgtagaatctacaagtggatatttggacctctctganggatttcgttggaaacgggataactgcacctaactaaacggaagcattcctcagaaacttctnggtgatgtttgcattcaaatcccanagtngaaccttcctttgaaagtncagggttgaannccnctttttgtagggatctgcangtggatnttgggaccctctgnggccttcgttcgaaacgggtatatctttcccannaaatntaaacagaagccttccccaaanctccccgggnagatgccnncncncaanagnnnancccccnttggaagangcaggtgaaaccctcttttgggaanncncaagggaattngnncccccccaaangtcttggaannggaatttccanaaaanaananancttccaaaactcccggggntnggnnacccnantncnntgttttnaaaattnaaannnttttcagggccaagggaattgggctccngcggggggaaaattggcccaaangggannnnccaacnttggngtccccaagaggncctnnaaanccccttgggcngggntccttattnggnnaaacnaanaann

SEQ ID NO: 2 - DNA, 1473 bp, Homo sapien
  misc_feature (107)..(107): n is a, c, g, or t
ccactgctgcctgcgaagtgtgtttctaaactgctacatcgcaaggaatgctcagctctgtgagttcaactcaatcatcccaaagaattttctgagaaagcttctgncttctttttataggaagttatttcctttactacggtactcctcaaagagtgcaatgatccccttgcagtttctacaaaaagagtgtttcaaacctgaactatcaaagaaaggttccacactgtgagttgaatgcagacatcacgaagaaggttctgagaatgcttctgtttagttctgtgcagtttatcccgtttccaacgaaatcctcagagaggaccaaatatccacttgcagtttctacaaaaagagtgtttcaaagctgaactatcaaagaaaggttcagcactgtgagttgaatgcaaacatcacgaagagggttctgagaatgcttctgttttagttctgtgcgggttatcccgtttccaacgaaatcctcagagcggtccaaatatctacttgcagtttctacagaaagaccgtttcaaacctgaactatcaaagaaaggttcaacactgtgagttgaatgcaaacatcacgaagaaggttctgagaatgcttctgtttagttctgtgcggtttatcccgtttccaacgaaatcctcagagaggcctaaatatccacttgcacattctacaaatagtgtgnttcgaaactgctccatccaaaggaatgttcagctctgtgagttaaactcagtcgtcaccaagagttttctgtgaatgctactgtctagcttttatatgaagctatttcctttactaccataggcctcaaagcggtcccatatctccacttgcagattctacacaaaagagagtttccaaactgctctgtcaaagggaatgttcaactctgtgactggaatgcaatcatcacnaaagtagtttctgagantgctnctatctagcttttacgggaagataattccttttccaccacanggcctcaangccctcnaatgtccncttgccagattctggaaaaagagngtttcaagcttctctctcgaaaggnangttcacctngggggttgaangcagcctcccaanaagtttctgaaaagctncggttagcttnccggganattcnccgttccacgaantttccaaanggnccaaantcccttgcaatccccnaaagangatgggancgcntttgaaaagaacntcacnngggggtggaagnancnccaaaanttncgannggcttcccccngtttntggacanattttttcccnngncnganncccaaggncctttgaancnccaangggttcaancccttaaaaagggactnggggtaacccccncnaattcgacctttttttttgaattccttcaacnnggggcnctcctntccaaaattnccccnanggcctnggaaacnaantggnttttttaanttaacgnnnntcnntcctnn